(19) United States
(12) Patent Application Publication
(10) Pub. No.: US 2024/0269258 A1
(43) Pub. Date: Aug. 15, 2024

(54) MULTIVALENT INFLUENZA VACCINES

(71) Applicant: SANOFI PASTEUR INC., Swiftwater, PA (US)

(72) Inventors: Timothy ALEFANTIS, Spring Brook Township, PA (US); Mario BARRO, Reading, MA (US); Anthony BYERS, Oviedo, FL (US); Philip DAVIDSON, Maynard, MA (US); Maryann GIEL-MOLONEY, Watertown, MA (US); Philippe-Alexandre GILBERT, Cary, NC (US); Harold KLEANTHOUS, Chelmsford, MA (US); Armaghan NAIK, Natick, MA (US); Konstantin PUGACHEV, Watertown, MA (US); Saranya SRIDHAR, London (GB); William WARREN, Orlando, FL (US); Konstantin ZELDOVICH, Belmont, MA (US)

(21) Appl. No.: 18/627,824

(22) Filed: Apr. 5, 2024

Related U.S. Application Data
(63) Continuation of application No. PCT/US2022/045992, filed on Oct. 7, 2022.
(60) Provisional application No. 63/253,986, filed on Oct. 8, 2021.
(60) Provisional application No. 63/277,848, filed on Nov. 10, 2021.

(51) Int. Cl. A61K 39/145; A61K 9/00; A61K 39/00; C07K 14/005

(52) U.S. Cl. CPC A61K 39/145; A61K 9/0019; C07K 14/005; A61K 2039/55555; A61K 2039/70; C12N 2760/16322; C12N 2760/16334

(57) ABSTRACT

Disclosed are multivalent vaccine or immunogenic compositions comprising influenza virus hemagglutinin (HA) from standard of care influenza virus strains, or ribonucleic acid molecules encoding the same; and one or more influenza virus HA identified or designed by machine learning, or one or more ribonucleic acid molecules that encode the influenza virus HA identified or designed by machine learning. Also disclosed are methods of using the vaccine or immunogenic compositions.

### CROSS REFERENCE TO RELATED APPLICATIONS

This application is a continuation application of International Patent Application No. PCT/US2022/045992, filed on 7 Oct. 2022, which claims the benefit of, and relies on the filing date of, U.S. Provisional Patent Application No. 63/253,986, filed 8 Oct. 2021, and U.S. Provisional Patent Application No. 63/277,848, filed 10 Nov. 2021, the entire disclosures of which are herein incorporated by reference.

### FIELD OF THE DISCLOSURE

Disclosed herein are multivalent influenza vaccine or immunogenic compositions comprising a plurality of influenza virus hemagglutinin (HA) proteins or ribonucleic acid molecules encoding influenza virus HA, wherein the multivalent vaccine or immunogenic composition includes at least one influenza virus HA (or ribonucleic acid molecule encoding the at least one influenza virus HA) having a molecular sequence identified or designed from a machine learning model. Further disclosed herein are methods of using the multivalent influenza vaccine or immunogenic compositions.

### BACKGROUND OF THE DISCLOSURE

Influenza is caused by a virus that attacks mainly the upper respiratory tract including the nose, throat and bronchi and rarely also the lungs. The infection usually lasts for about a week. It is characterized by sudden onset of high fever, myalgia, headache and severe malaise, non-productive cough, sore throat, and rhinitis. Most people recover within one to two weeks without requiring any medical treatment. However, in the very young, the elderly and people suffering from medical conditions, such as lung diseases, diabetes, cancer, kidney or heart problems, influenza poses a serious risk. In these people, the infection may lead to severe complications of underlying diseases, pneumonia, and death, although even healthy adults and older children can be affected as well. Annual seasonal influenza epidemics are thought to result in between three and five million cases of severe illness and between 250,000 and 500,000 deaths every year around the world.

Influenza virus is a member of the Orthomyxoviridae family. There are three main subtypes of influenza viruses, designated influenza A, influenza B, and influenza C. The influenza virion contains a segmented negative-sense RNA genome, which encodes the following proteins: hemagglutinin (HA), neuraminidase (NA), matrix (M1), proton ion-channel protein (M2), nucleoprotein (NP), polymerase basic protein 1 (PB1), polymerase basic protein 2 (PB2), polymerase acidic protein (PA), and nonstructural protein 2 (NS2). The HA, NA, M1, and M2 are membrane associated, whereas NP, PB1, PB2, PA, and NS2 are nucleocapsid associated proteins. The HA and NA proteins are envelope glycoproteins, primarily responsible for virus attachment and penetration of the viral particles into the cell and release from the cell, respectively.

Both HA and NA proteins are the sources of the major immunodominant epitopes for virus neutralization and protective immunity, making them important components for prophylactic influenza vaccines. The genetic makeup of influenza viruses allows frequent minor genetic changes, known as antigenic drift. Thus, the amino acid sequence of the major antigens of influenza, including HA and NA, is highly variable across certain groups, subtypes and/or strains. For this reason, current seasonal influenza vaccines are recommended every year and require yearly surveillance to account for mutations in HA (antigenic drift) and to match rapidly-evolving viral strains.

Certain known licensed influenza vaccine compositions are inactivated vaccines, containing entire virions or virions subjected to treatment with agents that dissolve lipids (“split” vaccines), purified glycoproteins expressed in cell culture (“sub-unit vaccines”), or live attenuated virus vaccines. Other types of vaccines are being developed, such as RNA/DNA based, viral vector based, etc. These vaccines offer protection by inducing production of a subject's antibodies directed against the antigens, e.g., HA. Antigenic evolution of the influenza virus by mutation results in modifications in HA and, to a lesser extent, NA. Accordingly, the available vaccines may only protect against strains having surface glycoproteins that comprise identical or cross-reactive epitopes. To provide a sufficient antigenic spectrum, conventional vaccines comprise components from several different viral strains, including strains from both Type A and Type B influenza. The choice of strains for use in vaccines is reviewed annually for each particular year and is predicated on World Health Organization (WHO) recommendations. These recommendations reflect international epidemiological observations.

The recommended WHO strains are known as standard of care strains and typically include an H1N1 subtype, an H3N2 subtype, a B/Yamagata lineage, and a B/Victoria lineage. As noted above, due to antigenic drift, the selection of standard of care strains must be updated every year in an attempt to match the anticipated circulating strains for that year. Thus, commercially available conventional influenza vaccines are typically quadrivalent vaccines including four HAs from influenza virus strains, one from each of H1 (H1N1), H3 (H3N2), B/Yamagata, and B/Victoria subtypes/lineages. The vaccine compositions may comprise recombinant HA proteins, inactivated virions such as split-inactivated virions, or attenuated virions. The WHO must select the standard of care strains well before the influenza season begins to give manufacturers sufficient time to produce the global vaccine supply, meaning that the standard of care strains selected by the WHO do not always match the circulating influenza strains for a particular year. Influenza vaccine effectiveness varies from about 40-60% depending on year and subtype, and is highly variable, especially for A/H3N2. Rapid antigenic drift of A/H3N2 has caused vaccine mismatches in the past, such as in the Northern Hemisphere 2018-2019 season. If the recommended standard of care strains selected by the WHO to be included in the seasonal vaccine preparations differ from a given season's circulating influenza strain or strains, the commercially available conventional influenza vaccine may provide reduced antigenic coverage and therefore lower protective efficacy against influenza disease.

Thus, the ability to supplement standard of care influenza strains in vaccines with an additional antigen or antigens that may confer added protection and/or protection against a wider variety of influenza strains and drifted HA strains is desirable.

### SUMMARY OF THE DISCLOSURE

The present disclosure provides a multivalent vaccine or immunogenic composition comprising influenza virus HAs from the standard of care influenza virus strains or ribonucleic acid molecules encoding the influenza virus HAs from standard of care influenza strains, and one or more machine learning influenza virus HA or ribonucleic acid molecules encoding the machine learning influenza virus HA. The one or more machine learning influenza virus HA (or ribonucleic acid encoding the same) may be selected to provide enhanced and/or broader breadth of protection against circulating influenza strains than the standard of care strains and increase vaccine effectiveness.

Disclosed herein is a vaccine or immunogenic composition comprising (a) at least three or at least four influenza virus HAs from standard of care influenza virus strains, or at least three or at least 4 ribonucleic acid molecule encoding the influenza virus HAs; and (b) one or more machine learning influenza virus HA having a molecular sequence identified or designed from a machine learning model, or one or more ribonucleic acid molecules encoding the one or more machine learning influenza virus HA. In certain embodiments, the one or more machine learning influenza virus HA are selected from an H1 HA, an H3 HA, an HA from a B/Victoria lineage, an HA from a B/Yamagata lineage, or a combination thereof.

In one aspect, disclosed herein is a vaccine or immunogenic composition comprising (a) a first influenza virus hemagglutinin (HA) wherein the first influenza virus HA is an H1 HA from a first standard of care influenza virus strain, or a first ribonucleic acid molecule encoding the first influenza virus H1 HA; (b) a second influenza virus HA wherein the second influenza virus HA is an H3 HA from a second standard of care influenza virus strain, or a second ribonucleic acid molecule encoding the second influenza virus H3 HA; (c) a third influenza virus HA wherein the third influenza virus HA is from a third standard of care influenza virus strain from the B/Victoria lineage, or a third ribonucleic acid molecule encoding the third influenza virus HA from the B/Victoria lineage; (d) a fourth influenza virus HA wherein the fourth influenza virus HA is from a fourth standard of care influenza virus strain from the B/Yamagata lineage, or a fourth ribonucleic acid molecule encoding the fourth influenza virus HA from the B/Yamagata lineage; and (e) one or more machine learning influenza virus HA having a molecular sequence identified or designed from a machine learning model, or one or more ribonucleic acid molecules encoding the one or more machine learning influenza virus HA, wherein the one or more machine learning influenza virus HA are selected from an H1 HA, an H3 HA, an HA from a B/Victoria lineage, an HA from a B/Yamagata lineage, or a combination thereof. Each of (and independently from the others, if any) the one or more HA having a molecular sequence identified or designed from a machine learning model, or each of (and independently from the others, if any) the one or more ribonucleic acid molecules encoding the one or more machine learning influenza virus HA may in certain aspects be antigenically dissimilar than, antigenically similar to, be from a different clade than, be from a same clade as, enhance a protective immune response induced by, and/or broaden a protective immune response induced by their respective standard of care influenza virus strain HA in the immunogenic composition.

In certain embodiments, the ribonucleic acid is an mRNA molecule, and in certain embodiments the ribonucleic acid molecule is encapsulated in a lipid-nanoparticle (LNP). In certain embodiments, the ribonucleic acid molecule is encapsulated in an LNP comprising a cationic lipid, a PEGylated lipid, a cholesterol-based lipid, and a helper lipid.

In various embodiments disclosed herein, the one or more machine learning influenza virus HA comprise a wild type influenza virus HA molecular sequence, and in certain embodiments, the machine learning influenza virus HA comprise a non-wild type influenza virus HA molecular sequence. In certain embodiments, the one or more machine learning influenza virus HA is a recombinant influenza virus HA, and in certain embodiments, the one or more machine learning influenza virus HA is present in an inactivated influenza virus, such as a split-inactivated virus. In certain embodiments the multivalent influenza vaccine comprises one or more ribonucleic acid molecules encoding at least one of the one or more machine learning influenza virus HA.

In various embodiments, the one or more machine learning influenza virus HA is a fifth influenza virus HA or a ribonucleic acid molecule encoding the fifth influenza virus HA, wherein the fifth influenza virus HA is an H3 HA. The fifth influenza virus H3 HA may in certain aspects be antigenically dissimilar than the second influenza H3 HA, antigenically similar to the second influenza H3 HA, enhance a protective immune response induced by the second influenza H3 HA, and/or broaden a protective immune response induced by the second influenza H3 HA. The fifth influenza virus H3 HA may in certain aspects be from a different clade than the second influenza H3 HA or may be from a same clade as the second influenza H3 HA. In certain embodiments, the fifth influenza virus H3 HA is from the 3C.2A clade, and in certain embodiments, the fifth influenza virus H3 HA is a from the 3C.3A clade.

In various embodiments, the one or more machine learning influenza virus HA is a fifth influenza virus HA or a ribonucleic acid molecule encoding the fifth influenza virus HA, wherein the fifth influenza virus HA is an H1 HA. The fifth influenza virus H1 HA may in certain aspects be antigenically dissimilar than the first influenza H1 HA, antigenically similar to the first influenza H1 HA, enhance a protective immune response induced by the first influenza H1 HA, and/or broaden a protective immune response induced by the first influenza H1 HA. The fifth influenza virus H1 HA may in certain aspects be from a different clade than the first influenza H1 HA or may be from a same clade as the first influenza H1 HA.

In certain embodiments of the vaccine or immunogenic compositions disclosed herein, the vaccine or immunogenic composition further comprises a sixth influenza virus HA. In certain embodiments, the sixth influenza virus HA is an H3 HA, and in certain embodiments, the sixth influenza virus is an H3 HA having a molecular sequence identified or designed from a machine learning model, or a ribonucleic acid molecule encoding the sixth influenza virus HA. In certain embodiments, the sixth influenza virus HA is an H1 HA, such as an H1 HA having a molecular sequence identified or designed from a machine learning model, or a ribonucleic acid molecule encoding the sixth influenza virus HA. In certain embodiments, the sixth influenza H1 HA is antigenically dissimilar than the first influenza H1 HA, enhances a protective immune response induced by the first influenza H1 HA, broadens a protective immune response induced by the first influenza H1 HA, is from a different clade than the first influenza H1 HA, is from a same clade as the first influenza H1 HA, or is antigenically similar to the first influenza H1 HA. In certain embodiments, the sixth influenza H3 HA is antigenically dissimilar than the second influenza H3 HA, enhances a protective immune response induced by the second influenza H3 HA, broadens a protective immune response induced by the second influenza H3 HA, is from a different clade than the second influenza H3 HA, is from a same clade as the second influenza H3 HA, or is antigenically similar to the second influenza H3 HA.

In certain embodiments, the vaccine or immunogenic composition disclosed herein further comprises a seventh influenza virus HA from the B/Victoria lineage or from the B/Yamagata lineage and having a molecular sequence identified or designed from a machine learning model, or a ribonucleic acid molecule encoding the seventh influenza virus HA.

In certain embodiments, the vaccine or immunogenic composition further comprises a seventh influenza virus HA from the B/Victoria lineage and an eighth influenza virus from the B/Yamagata lineage having a molecular sequence identified or designed from a machine learning model, or a ribonucleic acid molecule encoding the seventh and eighth influenza virus HA.

In certain aspects, the machine learning model is trained to predict a biological response, such as a human, ferret, or mouse biological response, and in certain aspects, the biological response comprises a hemagglutinin inhibition assay (HAI), antibody forensics (AF), or neutralization assay. In certain embodiments, the molecular sequence is an amino acid sequence or a nucleic acid sequence. In certain embodiments, the molecular sequence is an amino acid sequence.

In various aspects of the vaccine or immunogenic composition disclosed herein, each of the first, second, third, and fourth influenza virus HA is a recombinant influenza virus HA, such as recombinant influenza virus HA produced by a baculovirus expression system in cultured insect cells. In certain aspects, each of the first, second, third, and fourth influenza virus HA is present in an inactivated influenza virus, such as a split-inactivated virus. In still further aspects, the vaccine or immunogenic composition comprises the first, second, third, and fourth ribonucleic acid molecules as described herein.

In certain embodiments disclosed herein, the first influenza virus HA is an H1 HA from an H1N1 influenza virus strain and the second influenza virus HA is an H3 HA from an H3N2 influenza virus strain.

In certain embodiments, the vaccine or immunogenic composition further comprises an adjuvant, such as squalene-in-water adjuvant, such as AF03, or a liposome-based adjuvant, such as SPA14.

Another aspect of the disclosure is directed to methods of immunizing a subject against influenza virus, the method comprising administering to the subject an immunologically effective amount of the vaccine or immunogenic composition disclosed herein. Likewise the present disclosure provides an immunologically effective amount of a vaccine or immunogenic composition as described herein for use in immunizing a subject against influenza virus. Similarly, the present disclosure also provides the use of an immunologically effective amount of the vaccine or immunogenic composition as described herein for the manufacture of a medicament for immunizing against influenza virus. In certain embodiments, the method or use prevents influenza virus infection in the subject, and in certain embodiments, the method or use raises a protective immune response, such as an HA antibody response, in the subject. In certain embodiments of the methods or uses disclosed herein, the subject is human, and in certain embodiments, the human is 6 months of age or older, 6 to 35 months of age, at least 2 years of age, at least 3 years of age, less than 18 years of age, at least 18 years of age, at least 60 years of age, at least 65 years of age, at least 6 months of age and less than 18 years of age, at least 3 years of age and less than 18 years of age, or at least 18 years of age and less than 65 years of age. In certain embodiments, the vaccine or immunogenic composition is administered or prepared to be administered intramuscularly, intradermally, subcutaneously, intravenously, intranasally, by inhalation, or intraperitoneally. In certain embodiments, the method or use disclosed herein treats or prevents disease caused by either or both a seasonal and a pandemic influenza strain.

Also disclosed herein are methods of reducing one or more symptoms of influenza virus infection, the method comprising administering to a subject a prophylactically effective amount of the vaccine or immunogenic composition disclosed herein. Likewise the present disclosure provides a prophylactically effective amount of a vaccine or immunogenic composition as described herein for use in reducing one or more symptoms of influenza virus infection in a subject. Similarly, the present disclosure also provides the use of a prophylactically effective amount of the vaccine or immunogenic composition as described herein for the manufacture of a medicament for reducing one or more symptoms of influenza virus infection in a subject. In certain aspects, the methods or use disclosed herein comprise administering to the subject two doses of the vaccine or immunogenic composition with an interval of 2-6 weeks, optionally 4 weeks.

In another aspect, disclosed herein is a vaccine composition comprising the immunogenic composition disclosed herein.

### BRIEF DESCRIPTION OF THE FIGURES

FIG. 1 is a model illustration depicting a hypothetical example of virus samples Virus 1 and Virus 2 scored in an HAI assay, wherein the HAI titers of Virus 1 and Virus 2 may be compared to a previous season's vaccine virus to assess the antigenic similarity or dissimilarity of different virus strains.

FIG. 2 is a bar graph showing the average microneutralization titers for each group of ferrets co-infected with A/HONGKONG/45/2019 alone (grey), A/ALASKA/43/2019 alone (green), and a combination of A/HONGKONG/45/2019 and A/ALASKA/43/2019 (orange), as described in Example 1. The observed titers for seven strains of the 3C.2 clade are shown on the left, and the observed titers for five 3C.3 clade strains are shown on the right.

FIG. 3 is a bar graph showing the average microneutralization titers for each group of ferrets co-infected with A/HONGKONG/45/2019 alone (green), A/KANSAS/14/2017 alone (blue), and a combination of A/HONGKONG/45/2019 and A/KANSAS/14/2017 (orange), as described in Example 1. The observed titers for seven 3C.2 clade strains are shown on the left, and the observed titers for five 3C.3 clade strains are shown on the right.

FIG. 4A is a graph illustrating microneutralization titers for 3C.2 clade strains of influenza virus (top) and 3C.3 strains of influenza virus (bottom), after co-infection with A/HONGKONG/45/2019 alone (light grey), A/ALASKA/43/2019 alone (dark grey), and a combination of A/HONGKONG/45/2019 and A/ALASKA/43/2019 (orange), as described in Example 1.

FIG. 4B is a graph illustrating microneutralization titers for 3C.2 clade strains of influenza virus (top) and 3C.3 strains of influenza virus (bottom), after co-infection with A/HONGKONG/45/2019 alone (light grey), A/KANSAS/14/2017 alone (dark grey), and a combination of A/HONGKONG/45/2019 and A/KANSAS/14/2017 (orange), as described in Example 1.

FIG. 5 is a plot showing the average neutralization titers after co-infection with a combination of A/HONGKONG/45/2019 and A/ALASKA/43/2019 (blue) and after challenge with a combination of A/HONGKONG/45/2019 and A/KANSAS/14/2017 (orange) against the maximum average solo titers for each of the twelve strains evaluated, as described in Example 1.

FIG. 6 is graph illustrating the geometric mean titer (GMT) microneutralization assay titers for Groups 1-7 and 10 for each of A/Tasmania/503/2020, A/Victoria/2570/2019, B/Phuket/3073/2013, and B/Washington02/2019, as described in Example 2.

FIG. 7 is a bar graph showing the GMT microneutralization assay titers for Groups 1-7 and 10 for viruses in the 3C.2A clade (left bars for each Group) and viruses in the 3C.3A clade (right bars for each Group), as described in Example 2.

FIG. 8 is graph illustrating the geometric mean titer (GMT) microneutralization assay titers for Groups 1-7 and 10 for each of A/Bangladesh/3190613015/2019, A/Hong Kong/45/2019, A/Singapore/INFIMH160019/2016, A/Valladolid/182/2017, A/Kansas/14/2017, and A/Mexico/2356/2019, as described in Example 2.

FIG. 9 is a bar graph showing the percent coverage (>1:160 GMT value) for each of Groups 1-7 for viruses in the 3C.2A clade (left bars for each Group) and viruses in the 3C.3A clade (right bars for each Group), as described in Example 2.

### DETAILED DESCRIPTION OF THE DISCLOSURE

Some viruses are capable of substantial variation in the structure of their envelope glycoprotein components. Influenza virus, for example, constantly changes the amino acid sequence of its envelope glycoproteins. Either major amino acid variations (antigenic shift) or minor variations (antigenic drift) can give rise to new epitopes, allowing the virus to evade the immune system. The antigenic variation is the major cause of repeated influenza outbreaks. Antigenic variants within a subtype (i.e., H1 or H3) emerge and are gradually selected as predominant virus while the preceding virus is suppressed by specific antibody arising in the population. Neutralizing antibody to one variant generally becomes less and less effective as sequential variants arise. The immune response to variants within a subtype may depend on the prior experience of the host.

The rate of silent nucleotide substitution has been shown to be higher than the rate of coding nucleotide substitutions for all genes of influenza virus, including the gene for HA (Reviewed by Webster et al.; Webster, R. G., et al., 1992). However, HA has a much higher rate of coding changes than the internal proteins. The elevated rate of coding nucleotide changes in the HA gene as compared with other genes has been taken as evidence that immune selection is an important factor in its evolution (Palese, P., et al., 1982). Using reassorted antigens to eliminate any nonspecific steric hindrance, Kilbourne et al. studied the rate of evolution of epidemiologically important HA and NA antigens isolated from humans over a 10-year period and determined that the HA evolved more rapidly than the neuraminidase (NA) (Kilbourne, E. D., et al., 1990). This was shown with both type A H1N1 and H3N2 viruses and has been confirmed by subsequent experiments with more recent strains. The reason for the apparently different rates of evolution is unknown but may be due to the fact that antibody to HA neutralizes virus and prevents infection. This places more selective pressure on the HA to maintain itself in a partially immune population.

Thus, there may be an increase of vaccine effectiveness with an addition of HA antigens derived from supplemental strains. This increase in efficacy may be due to two primary mechanisms. First, inclusion of one or more additional HA antigens may allow protection against a broader range of circulating influenza strains, for example if the circulating strains are matched or are antigenically similar to the additional strain(s) but not to the standard of care strains. Second, for the circulating strains that are antigenically similar to both a standard of care strain and an additional strain, the dose of matching or similar antigens in the vaccine may be effectively doubled, in turn increasing antibody titer and seroconversion rate. Either or both mechanisms may increase vaccine effectiveness.

Accordingly, disclosed herein are multivalent influenza vaccines comprising, in addition to influenza virus HA derived from standard of care influenza virus strains (and/or ribonucleic acid molecules encoding such standard of care influenza virus HA), one or more supplemental HA proteins or ribonucleic acid molecules encoding the same that may be identified or designed using a machine learning model.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Adjuvant: As used herein, the term “adjuvant” refers to a substance or combination of substances that may be used to enhance an immune response to an antigen component of a vaccine.

Antigen: As used herein, the term “antigen” refers to an agent that elicits an immune response and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, ferrets, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. Antigens include the HA forms as described herein.

Antigenically dissimilar: As used herein, the term “antigenically dissimilar” indicates that two antigens (e.g., HA antigens) generate an antibody response, as measured by binding titers or neutralizing titers, that is greater than 4-fold from each other, as described below. HA antigens from different clades may be antigenically dissimilar.

Antigenically similar: As used herein, the term “antigenically similar” indicates that two antigens generate an antibody response, as measured by binding titers or neutralizing titers, that is within 4-fold of each other, as described below.

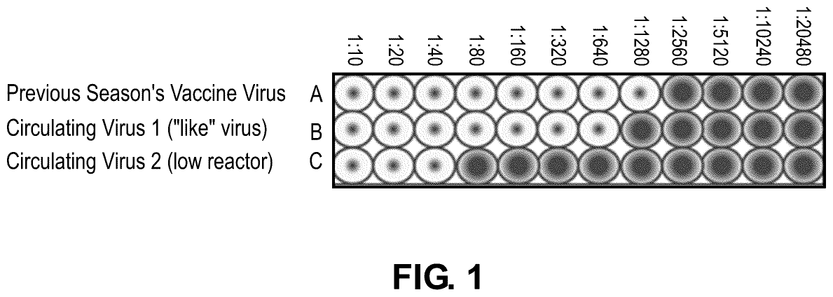

To assess whether two antigens are antigenically dissimilar or antigenically similar a naïve ferret model may be used as described in the examples. In this model, naïve ferrets are intranasally infected with a live influenza virus, and sera collected to assess an antibody response to the virus. Antibody responses may be measured by a hemagglutinin inhibition (HAI) assay measuring virus antibody binding titers, or by a neutralization assay (e.g., microneutralization assay) measuring virus neutralization titers. The efficiency of binding or neutralizing heterologous viral strains can indicate whether strains are antigenically dissimilar or antigenically similar. FIG. 1 illustrates virus samples scored in an HAI assay. When circulating Virus 1 is compared to the vaccine virus, the circulating Virus 1 differs by one dilution (a 2-fold difference) and, therefore, is considered antigenically similar to the previous season's vaccine virus. When circulating Virus 2 is compared to the vaccine virus, the circulating Virus 2 differs by 5 dilutions (a 32-fold difference) and, therefore, is considered antigenically dissimilar from the previous season's vaccine virus.

Approximately: As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Epitope: As used herein, the term “epitope” includes any moiety that is specifically recognized by an immunoglobulin (e.g., antibody or T cell receptor) binding component in whole or in part. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms or groups are physically separated from one another when the antigen adopts an alternative conformation (e.g., is linearized).

Excipient: As used herein, the term “excipient” refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Immune response: As used herein, the term “immune response” refers to a response of a cell of the immune system, such as a B cell, T cell, dendritic cell, macrophage or polymorphonucleocyte, to a stimulus such as an antigen, immunogen, or vaccine. An immune response can include any cell of the body involved in a host defense response, including for example, an epithelial cell that secretes an interferon or a cytokine. An immune response includes, but is not limited to, an innate and/or adaptive immune response. Methods of measuring immune responses are well known in the art and include, for example, measuring proliferation and/or activity of lymphocytes (such as B or T cells), secretion of cytokines or chemokines, inflammation, antibody production and the like. An antibody response or humoral response is an immune response in which antibodies are produced. A “cellular immune response” is one mediated by T cells and/or other white blood cells.

Immunogen: As used herein, the term “immunogen” or “immunogenic” refers to a compound, composition, or substance which is capable, under appropriate conditions, of stimulating an immune response, such as the production of antibodies or a T cell response in an animal, including compositions that are injected or absorbed into an animal. As used herein, “immunize” means to induce in a subject a protective immune response against an infectious disease (e.g., influenza).

Immunologically effective amount: As used herein, the term “immunologically effective amount” means an amount sufficient to immunize a subject.

In some embodiments: As used herein, the term “in some embodiments” refers to embodiments of all aspects of the disclosure, unless the context clearly indicates otherwise.

Machine learning: As used herein, the term “machine learning” refers to the use of algorithms that improve automatically through experience and/or by the use of data. Machine learning may involve construction of a predictive model, such as a model of influenza antigenicity, to allow prediction of data, including the use of an algorithm designed to select candidate antigens through the predictive model. Target strains may be identified and a selection algorithm may then be constructed. Examples of machine learning algorithms and methods can be found, for example, in PCT Application Nos. WO 2021/080990 A1, entitled Systems and Methods for Designing Vaccines, and WO 2021/080999 A1, entitled Systems and Methods for Predicting Biological Responses, both of which are incorporated by reference in their entireties herein. Machine learning, as used herein, may also include the application of computation tools to analyze and interpret data, for example, bioinformatics analyses, such as phylogenetic analysis. Likewise, a “machine learning influenza virus HA” indicates an influenza virus HA that has been identified or designed by machine learning. A “machine learning model” indicates a model that uses algorithms that improve automatically through experience and/or by the use of data in order to predict data, such as a candidate antigen.

Pandemic strain: A “pandemic” influenza strain is one that has caused or has capacity to cause pandemic infection of subject populations, such as human populations. In some embodiments, a pandemic strain has caused pandemic infection. In some embodiments, such pandemic infection involves epidemic infection across multiple territories; in some embodiments, pandemic infection involves infection across territories that are separated from one another (e.g., by mountains, bodies of water, as part of distinct continents, etc.) such that infections ordinarily do not pass between them.

Prevention: The term “prevention”, as used herein, refers to prophylaxis, avoidance of disease manifestation, a delay of onset, and/or reduction in frequency and/or severity of one or more symptoms of a particular disease, disorder or condition (e.g., infection for example with influenza virus). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Recombinant: As used herein, the term “recombinant” is intended to refer to polypeptides (e.g., HA polypeptides as described herein) that are designed, engineered, prepared, expressed, created or isolated by recombinant means, such as polypeptides expressed using a recombinant expression vector transfected into a host cell, polypeptides isolated from a recombinant, combinatorial polypeptide library or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more of such selected sequence elements results from mutagenesis (e.g., in vivo or in vitro) of a known sequence element, e.g., from a natural or synthetic source. In some embodiments, one or more of such selected sequence elements results from the combination of multiple (e.g., two or more) known sequence elements that are not naturally present in the same polypeptide (e.g., two epitopes from two separate HA polypeptides).

Seasonal strain: A “seasonal” influenza strain is one that has caused or has capacity to cause a seasonal infection (e.g., annual epidemic) of subject populations, such as human populations. In some embodiments, a seasonal strain has caused seasonal infection.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. “Sequence identity” between two nucleic acid sequences indicates the percentage of nucleotides that are identical between the sequences. “Sequence identity” between two amino acid sequences indicates the percentage of amino acids that are identical between the sequences. Homologs or variants of a given gene or protein will possess a relatively high degree of sequence identity when aligned using standard methods.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90%, at least about 95%, at least about 96%, at least about 97%, at least about 98%, at least about 99%, or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional and/or structural property of said given sequence, e.g., and in some instances, are functionally and/or structurally equivalent to said given sequence. In some embodiments, a nucleic acid sequence or amino acid sequence having a particular degree of identity to a given nucleic acid sequence or amino acid sequence is functionally and/or structurally equivalent to said given sequence.

Standard of Care Strain: Each year, based on intensive surveillance efforts, the World Health Organization (WHO) selects influenza strains to be included in the seasonal vaccine preparations. As used herein, the term “standard of care strain” or “SOC strain” refers to an influenza strain that is selected by the World Health Organization (WHO) to be included in the seasonal vaccine preparations, for example for the Northern and Southern hemispheres. A standard of care strain can include a historical standard of care strain, a current standard of care strain or a future standard of care strain.

Subject: As used herein, the term “subject” means any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In some embodiments, the non-human subject is a mammal (e.g., a rodent, a mouse, a rat, a rabbit, a ferret, a monkey, a dog, a cat, a sheep, cattle, a primate, and/or a pig). In some embodiments, a subject may be a transgenic animal, genetically-engineered animal, and/or a clone. In some embodiments, the subject is an adult, an adolescent or an infant. In some embodiments, terms “individual” or “patient” are used and are intended to be interchangeable with “subject.”

Vaccine composition: As used herein, the term “vaccine composition” or “vaccine” refers to a composition that generates a protective immune response in a subject. As used herein, a “protective immune response” refers to an immune response that protects a subject from infection (prevents infection or prevents the development of disease associated with infection) or reduces the symptoms of infection (for instance an infection by an influenza virus). Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous, intraperitoneal, intradermal, or intramuscular. Vaccines may be administered with an adjuvant to boost the immune response.

Immunogenic composition: As used herein, the term “immunogenic composition” refers to a composition that generates an immune response that may or may not be a protective immune response.

Vaccinate: As used herein, the term “vaccinate” or the like refers to the administration of a vaccine composition to generate a protective immune response in a subject, for example to a disease-causing agent such as an influenza virus. Vaccination can occur before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccine composition.

Wild type (WT): As is understood in the art, the term “wild type” generally refers to a normal form of a protein or nucleic acid, as is found in nature. For example, wild type HA polypeptides are found in natural isolates of influenza virus. A variety of different wild type HA sequences can be found in the NCBI influenza virus sequence database.

### Nomenclature for Influenza Virus

All nomenclature used to classify influenza virus is that commonly used by those skilled in the art. Thus, a Type, or Group, of influenza virus refers to the three main types of influenza: influenza Type A, influenza Type B or influenza Type C that infect humans. Influenza A and B cause significant morbidity and mortality each year. It is understood by those skilled in the art that the designation of a virus as a specific Type relates to sequence difference in the respective M1 (matrix) protein or P (nucleoprotein). Type A influenza viruses are further divided into group 1 and group 2. These groups are further divided into subtypes, which refers to classification of a virus based on the sequences of two proteins on the surface of the virus HA and NA.

Currently, there are 18 recognized HA subtypes (H1-H18) and 11 recognized NA subtypes (N1-N11). Group 1 contains N1, N4, N5, and N8 and H1, H2, H5, H6, H8, H9, H11, H12, H13, H16, H17 and H18. Group 2 contains N2, N3, N6, N7, and N9 and H3, H4, H7. H10, H14, and H15. N10 and N11 have been identified in influenza-like genomes isolated from bats (Wu et al., Trends in Microbiology, 2014, 22(4): 183-91). While there are potentially 198 different influenza A subtype combinations, only about 131 subtypes have been detected in nature. Current subtypes of influenza A viruses that commonly circulate in the human population, giving rise to seasonal outbreaks, include: A(H1N1) and A(H3N2).

For convenience, certain abbreviations can be used to refer to protein constructs, and portions thereof, described herein. For example, HA can refer to an influenza hemagglutinin protein. H1 refers to HA from an influenza subtype 1 strain. H3 refers to HA from an influenza subtype 3 strain.

Influenza A subtypes can be further broken down into different genetic “clades” and “sub-clades.” For example, A subtype A(H1N1) contains clade 6B.1 and sub-clade 6B.1A. A subtype A(H3N2) contains clades 3C.2A and 3C.3A and sub-clades 3C.2A1, 3C.2A2, 3C2A3, and 3C.2A4. Likewise, B subtype Victoria contains clade V1A and sub-clades V1A.1, V1A.2, and V1A.3. while B subtype Yamagata contains clades Y1, Y2, and Y3. Finally, the term strain refers to viruses within a subtype that differ from one another in that they have small, genetic variations in their genome.

### Hemagglutinin (HA)

Hemagglutinin (HA), along with neuraminidase (NA), is one of the two major influenza surface proteins. The function of HA involves interactions with sialic acid, a terminal molecule bound to sugar moieties on glycoproteins or glycolipids expressed on the surface of cells. The binding of HA to sialic acid on the cell surface induces endocytosis of the virus by the cell, allowing the virus to gain entry and infect cells. Sialic acid is also added to HA as part of the glycosylation process that occurs within infected cells.

HA is believed to mediate attachment of the influenza virus to the host cell and viral-cell membrane fusion during penetration of the virus into the cell. Antigenic variation in the HA molecule is responsible for frequent outbreaks to influenza and for limited control of infection by immunization.

HA is present in mature influenza virus as trimers. Each HA monomer consists of two polypeptides (HA1 and HA2) linked by a disulfide bond. These polypeptides are derived by cleavage of a single precursor protein, HA0, during maturation of the influenza virus. In part, because these molecules are tightly folded, the HA0 and the mature HA1 and HA2 differ slightly in their conformation and antigenic characteristics. Furthermore, the HA0 is more stable and resistant to denaturation and to proteolysis. Baculovirus/insect cell cultures derived recombinant HA0 is known to confer protective immunity to influenza.

The influenza virus HAs present in the vaccine or immunogenic compositions disclosed herein may be any form of influenza virus HA and may include any combination of HA from standard of care influenza virus strains and machine learning influenza virus HA. For example, in certain embodiments the influenza virus HA from a standard of care influenza strain may be present in a vaccine or immunogenic composition as HA present in an inactivated influenza virus, recombinant influenza virus HA, or ribonucleic acid molecules encoding the aforementioned influenza virus HAs, or any combination thereof. In certain additional embodiments, the one or more machine learning influenza virus HA may be present in a vaccine or immunogenic composition as HA present on an inactivated influenza virus, recombinant influenza virus HA, ribonucleic acid molecules encoding the aforementioned machine learning influenza virus HAs, or a combination thereof.

Likewise, in the embodiments disclosed herein, the influenza virus HAs from the standard of care influenza strains and the HAs identified or designed from machine learning may be wild-type HA, non-wild type HA, HA from seasonal or pandemic influenza virus strains, and/or HA in any other form known in the art. In certain embodiments disclosed herein, the influenza virus HA is from a pandemic strain or a strain with pandemic potential, including, for example, H1, H2, H3, H5, H7, and/or H10.

In certain embodiments disclosed herein, the HA from a standard of care influenza virus strain and/or the machine learning influenza virus HA is present in an inactivated influenza virus.

Certain licensed influenza vaccines may comprise formalin-inactivated whole or chemically split subunit preparations from multiple influenza subtypes, including, for example, influenza A subtype H1N1, influenza A H3N2, influenza B/Victoria, and/or influenza B/Yamagata. The seed viruses for such influenza A and B vaccines may be naturally occurring strains (i.e.., wild-type strains) that replicate to high titers in the allantoic cavity of chicken eggs or cultured cells.

Alternatively, the strains may be a reassortant virus with the correct surface antigen genes. A reassortant virus is one that, due to segmentation of the viral genome, has characteristics of each parental strain. When more than one influenza viral strain infects a cell, these viral segments mix to create progeny virion containing various assortments of genes from both parents. The reverse genetics methods used to produce infectious, reassortant viruses are well-known by the one skilled in the art and include, but are not limited to, the methods using the plasmids described in Neuman et al, 1999, Proc Natl Acad Sci USA, 96(16): 9345-9350; Neumann et al, 2005, Proc Natl Acad Sci USA, 102(46): 16825-16829; Zhang et al, 2009, J Virol, 83(18): 9296-9303; Massin et al, 2005, J Virol, 79(21): 1381 1-13816; Murakami et al, 2008, 82(3): 1605-1609; and/or the cells described in Neuman et al, 1999, Proc Natl Acad Sci USA, 96(16): 9345-9350; Neumann et al, 2005, Proc Natl Acad Sci USA, 102(46): 16825-16829; Zhang et al, 2009, J Virol, 83(18): 9296-9303; Massin et al, 2005, J Virol, 79(21): 1381 1-13816; Murakami et al, 2008, 82(3): 1605-1609; Koudstaal et al, 2009, Vaccine, 27(19): 2588-2593; Schickli et al, 2001, Philos Trans R Soc Lond Biol Sci, 356(1416): 1965-1973; Nicolson et al, 2005, Vaccine, 23(22): 2943-2952; Legastelois et al, 2007, Influenza Other Respi Viruses, 1 (3): 95-104; Whiteley et al, 2007, Influenza Other Respi Viruses, 1 (4): 157-166.

Accordingly, the HA proteins disclosed herein include HA present in inactivated virions. In certain embodiments. the inactivated virus is a split inactivated virus. In certain embodiments, disclosed herein is an influenza virus HA present in an inactivated virus, wherein the HA is selected from an H1 HA from a standard of care influenza virus, an H3 HA from a standard of care influenza virus, an HA from a standard of care influenza virus strain from the B/Victoria lineage, or an HA from a standard of care influenza virus from the B/Yamagata lineage.

In certain embodiments, disclosed herein is a machine learning influenza virus HA, wherein the HA is present in the inactivated virus, and wherein the machine learning HA is selected from one or more of H1 HA, H3 HA, HA from a B/Victoria lineage, HA from a B/Yamagata lineage, or combinations thereof.

Also disclosed herein are vaccine or immunogenic compositions comprising recombinant HA, including recombinant HA from a standard of care influenza virus strain and/or machine learning recombinant HA.

Isolation, propagation and purification of influenza viral strains in order to clone the desired HA genes may be performed by any method known in the art, including, for example, those disclosed in U.S. Pat. No. 5,762,939, incorporated by reference herein.

Recombinant HA antigens are expressed in cells, such as insect cells, infected with viral-hemagglutinin vectors. The primary gene product is unprocessed, full-length HA (rHA0) and is not secreted but remains associated with peripheral membranes of infected cells. In insect cells, this rHA0 is glycosylated with N-linked, high-mannose type glycans, and there is evidence that rHA0 forms trimers post-translationally, which then accumulate in cytoplasmic cell membranes.

rHA0 can be selectively extracted from the peripheral membranes with a non-denaturing, non-ionic detergents or other methods known in the art for the purification of recombinant proteins from cell, e.g., insect cells, including, for example, affinity or gel chromatography, antigen binding, DEAE ion exchange, or lentil lectin affinity chromatography. The purified rHA0 may then be resuspended in an isotonic, buffered solution. In certain embodiments, the rHA0 is purified to at least about 80%, such as at least about 85%, at least about 90%, at least about 95%, at least about 96%, at least about 97%, at least about 98%, or at least about 99%.

In certain embodiments, full-length, uncleaved (HA0) hemagglutinin antigen from an influenza virus may be produced with baculovirus expression vectors in cultured insect cells and can be further purified, for example, under non-denaturing conditions. Two or more (such as three, four or more) purified hemagglutinin antigens from influenza A and/or influenza B strains may be mixed together to produce a multivalent influenza vaccine.

Baculoviruses are DNA viruses in the family Baculoviridae. These viruses are known to have a narrow host-range that is limited primarily to the Lepidopteran species of insects (e.g., butterflies and moths). For example, the baculovirus Autographa californica Nuclear Polyhedrosis Virus (AcNPV) replicates efficiently in susceptible cultured insect cells. AcNOV has a double-stranded closed circular DNA genome of about 130,000 base pairs and is well-characterized with regard to host range, molecular biology, and genetics.

Many baculoviruses, including AcNPV, form large protein crystalline occlusions within the nucleus of infected cells. A single polypeptide, referred to as a polyhedrin, accounts for approximately 95% of the protein mass of these occlusion bodies. The gene for polyhedrin is present as a single copy in the AcNPV viral genome. Because the polyhedrin gene is not needed for virus replication in culture cells, it can be readily modified to express foreign genes. The foreign gene sequence may be inserted into the AcNPV gene just 3′ to the polyhedrin promotor sequence such that it is under the transcriptional control of the polyhedrin promoter. Recombinant baculoviruses, including recombinant baculoviruses encoding recombinant HA proteins, may then replicate in a variety of insect cell lines. Recombinant HA proteins may also be expressed in other expression vectors, including, for example, Entomopox viruses (the poxviruses of insects), cytoplasmic polyhedrosis viruses (CPV), and transformation of insect cells with the recombinant HA gene or genes.

### Ribonucleic Acid Molecules Encoding HA

Also disclosed herein are ribonucleic acid molecules, such as mRNA molecules, that encode one or more of the influence virus HA disclosed herein. The ribonucleic acid molecules such as mRNA may encode a standard of care influenza virus strain, such as any one of a combination of an H1 HA, H3 HA, HA from a B/Victoria lineage, or HA from a B/Yamagata lineage. In certain embodiments, the ribonucleic acid molecules such as mRNA may encode a machine learning influenza virus HA, such as any one of a combination of an H1 HA, H3 HA, HA from a B/Victoria lineage, or HA from a B/Yamagata lineage. In certain embodiments, the ribonucleic acid molecule is encapsulated in a lipid-nanoparticle (LNP).

Exemplary mRNA and LNP are disclosed, for example, in International Application No. PCT/US2021/058250, filed Nov. 5, 2021, which is incorporated by reference in its entirety.

Any known LNP formulations may be used in the embodiments disclosed herein. In certain embodiments, the LNPs comprise a mixture of four lipids: an ionizable (e.g., cationic) lipid, a polyethylene glycol (PEG)-conjugated lipid, a cholesterol-based lipid, and a helper lipid, such as a phospholipid. The LNPs are used to encapsulate ribonucleic acid molecules (e.g., mRNA). The encapsulated mRNA molecules can be comprised of naturally-occurring ribonucleotides, chemically-modified nucleotides, or a combination thereof, and can each or collectively code for one or more proteins.

The ionizable lipid facilitates mRNA encapsulation and may be a cationic lipid. A cationic lipid affords a positively charged environment at low pH to facilitate efficient encapsulation of the negatively charged mRNA drug substance.

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or C₁₈) length, such as a derivatized ceramide (e.g., N-octanoyl-sphingosine-1-[succinyl(methoxypolyethylene glycol)] (C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); 1,2-distcaroyl-rac-glycero-polyethelene glycol (DSG-PEG); N,N ditetradecylacetamide-polyethylene glycol (e.g., ALC-0159); or 1-monomethoxypolyethyleneglycol-2.3-dimyristylglycerol (e.g., PEG2000-DMG).

The PEG preferably has a high molecular weight, e.g., 2000-2400 g/mol. In some embodiments, the PEG is PEG2000 (or PEG-2K). In particular embodiments, the PEGylated lipid herein is DMG-PEG2000, DSPE-PEG2000, DLPE-PEG2000, DSG-PEG2000, or C8 PEG2000. The PEGylated lipid component provides control over particle size and stability of the nanoparticle. The addition of such components may prevent complex aggregation and provide means for increasing circulation lifetime and increasing delivery of the lipid-nucleic acid pharmaceutical composition to target tissues (Klibanov et al., FEBS Letters (1990) 268 (1): 235-7). These components may be selected to rapidly exchange out of the pharmaceutical composition in vivo (see, e.g., U.S. Pat. 5,885,613).

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), 1,4-bis(3-N-oleylamino-propyl)piperazine (Gao et al., Biochem Biophys Res Comm. (1991) 179: 280; Wolf et al., BioTechniques (1997) 23: 139; U.S. Pat. No. 5,744,335), imidazole cholesterol ester (“ICE”; WO 2011/068810), B-sitosterol, fucosterol, stigmasterol, and other modified forms of cholesterol. In some embodiments, the cholesterol-based lipid used in the LNPs is cholesterol.

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. In certain embodiments, the helper lipid is a phospholipid. Examples of helper lipids are 1,2-dioleoyl-SN-glycero-3-phosphoethanolamine (DOPE); 1.2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC). dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), diolcoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a combination thereof.

In certain embodiments disclosed herein, the LNP comprises (i) a cationic lipid selected from OF-02, cKK-E10, GL-HEPES-E3-E10-DS-3-E18-1, GL-HEPES-E3-E12-DS-4-E10, GL-HEPES-E3-E12-DS-3-E14, ALC-0315, or SM-102; (ii) DMG-PEG2000; (iii) cholesterol; and (iv) DOPE.

In certain embodiments disclosed herein, the LNP comprises (i) ALC-0315 as the cationic lipid, (ii) N,N ditetradecylacetamide-polyethylene glycol (e.g., ALC-0159) as the PEGylated lipid, (iii) DSPC as the helper lipid, and (iv) cholesterol. In certain embodiments, the LNP comprises (i) ALC-0315 as the cationic lipid at a molar ratio of about 25% to about 65%, for example about 46.3%; (ii) N,N ditetradecylacetamide-polyethylene glycol (e.g., ALC-0159) as the PEGylated lipid at a molar ratio of about 0.5% to about 2.6%, for example 1.6%, (iii) DSPC as the helper lipid at a molar ratio of about 5% to about 15%, for example 9.4%, and (iv) cholesterol at a molar ratio of about 20% to about 60%, for example 42.7%.

The molar ratios of the above LNP components may assist in the LNPs' effectiveness in delivering mRNA. The molar ratio of the cationic lipid, the PEGylated lipid, the cholesterol-based lipid, and the helper lipid is A:B:C:D, wherein A+B+C+D=100%. In some embodiments, the molar ratio of the cationic lipid in the LNPs relative to the total lipids (i.e., A) is 35-50%. In some embodiments, the molar ratio of the PEGylated lipid component relative to the total lipids (i.e., B) is 0.25-2.75%. In some embodiments, the molar ratio of the cholesterol-based lipid relative to the total lipids (i.e., C) is 20-50%. In some embodiments, the molar ratio of the helper lipid relative to the total lipids (i.e., D) is 5-35%. In some embodiments, the (PEGylated lipid+cholesterol) components have the same molar amount as the helper lipid. In some embodiments, the LNPs contain a molar ratio of the cationic lipid to the helper lipid that is more than 1.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

In particular embodiments, the LNPs contain a cationic lipid, a PEGylated lipid, a cholesterol-based lipid, and a helper lipid at a molar ratio of 40:1.5:28.5:30. In further specific embodiments, the LNPs contain (i) OF-02, cKK-E10, GL-HEPES-E3-E10-DS-3-E18-1, GL-HEPES-E3-E12-DS-4-E10, or GL-HEPES-E3-E12-DS-3-E14; (ii) DMG-PEG2000; (iii) cholesterol; and (iv) DOPE at 40:1.5:28.5:30.

Where desired, the LNP or the LNP formulation may be multi-valent. In some embodiments, the LNP may carry ribonucleic acid molecules (e.g., mRNA) that encode more than one antigen, such as two, three, four, five, six, seven, eight, nine, ten, or more antigens, from the same or different pathogens. For example, the LNP may carry multiple ribonucleic acid molecules (e.g., mRNA), each encoding a different antigen; or carry a polycistronic mRNA that can be translated into more than one antigen (e.g., each antigen-coding sequence is separated by a nucleotide linker encoding a self-cleaving peptide such as a 2A peptide). An LNP carrying different ribonucleic acid molecules (e.g., mRNA) typically comprises (encapsulate) multiple copies of each mRNA molecule. For example, an LNP carrying or encapsulating two different ribonucleic acid molecules (e.g., mRNA) typically carries multiple copies of each of the two different ribonucleic acid molecules (e.g., mRNA).

In some embodiments, a single LNP formulation may comprise multiple kinds (e.g., two, three, four, five, six, seven, eight, nine, ten, or more) of LNPs, each kind carrying a different ribonucleic acid molecule (e.g., mRNA).

In some embodiments, the vaccine or immunogenic composition disclosed herein comprises ribonucleic acid molecules encoding polypeptides derived from one or more (e.g., two, three, four, five, six, seven, eight, nine, or ten) influenza viral proteins selected from H1 HA, H3 HA, HA from a B/Victoria lineage, and/or HA from a B/Yamagata lineage. In further embodiments, the vaccine or immunogenic compositions disclosed herein contain four ribonucleic acid molecules (e.g., mRNA). wherein a first ribonucleic acid molecule encodes an H1 HA from a first standard of care influenza virus strain, a second ribonucleic acid molecule encodes an H3 HA from a second standard of care influenza virus strain, a third ribonucleic acid molecule encodes an HA from a third standard of care influenza virus strain from the B/Victoria lineage, and a fourth ribonucleic acid molecule encodes an HA from a fourth standard of care influenza virus strain from the B/Yamagata lineage. In certain embodiments, the vaccine or immunogenic composition further comprises one or more ribonucleic acid molecules (e.g., mRNA) encoding the one or more machine learning influenza virus HA as disclosed herein, wherein the one or more machine learning influenza virus HA are selected from an H1 HA, an H3 HA, an HA from a B/Victoria lineage, an HA from a B/Yamagata lineage, or a combination thereof.

In certain embodiments, the vaccine or immunogenic compositions disclosed herein may comprise one or more self-amplifying ribonucleic acids, such as one or more self-amplifying mRNA encoding an influenza virus HA. Antigen expression from traditional mRNA is proportional to the number of mRNA molecules successfully delivered to a subject from a vaccine or immunogenic composition. Self-amplifying mRNA, however, comprise genetically-engineered replicons derived from self-replicating viruses, and therefore may be added to a vaccine or immunogenic composition in lower dosages than traditional mRNA while achieving comparable results.

The self-amplifying mRNA may encode any of the influenza virus HAs disclosed herein, including, for example, an H3 HA from a standard of care influenza virus, an H1 HA from a standard of care influenza virus, an HA from a standard of care influenza virus from the B/Victoria lineage, an HA from a standard of care influenza virus from the B/Yamagata lineage, and/or one or more machine learning influenza virus HA.

The ribonucleic acid molecule (e.g., mRNA) may be unmodified (i.e., containing only natural ribonucleotides A, U, C, and/or G linked by phosphodiester bonds), or chemically modified (e.g., including nucleotide analogs such as pseudouridines (e.g., N-1-methyl pseudouridinc), 2′-fluoro ribonucleotides, and 2′-methoxy ribonucleotides, and/or phosphorothioate bonds). The ribonucleic acid molecule (e.g., mRNA) may comprise a 5′ cap and a polyA tail. In certain embodiments, the one or more ribonucleic acid molecules comprises one or more modified nucleotides, and in certain embodiments, the one or more modified nucleotides are selected from pseudouridine, methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thiopseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine. In an embodiment, the modified nucleotides are methylpseudouridine, in particular 1N-methylpseudouridine. In certain embodiments, every uridine in the ribonucleic acid molecule is replaced by a pseudouridine, e.g., a methylpseudouridine, such as 1N-methylpseudouridine.

Each ribonucleic acid molecule may be present in the compositions disclosed herein in an amount effective to induce an immune response in a subject to which the composition is administered. In certain embodiments, each ribonucleic acid molecule may be present in the vaccine or immunogenic compositions disclosed herein in an amount ranging, for example, from about 0.1 μg to about 150 μg, such as from about 5 μg to about 120 μg, from about 10 μg to about 60 μg, or about 15 μg to about 45 μg. In certain embodiments, each ribonucleic acid molecule is present in the vaccine or immunogenic composition in an amount sufficient to encode, for example, from about 5 μg to about 120 μg, such as from about 10 μg to about 60 μg, or about 15 μg to about 45 μg of the influenza virus HA.

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands. stabilizing reagents (e.g., preservatives and antioxidants), and/or other pharmaceutically acceptable excipients. Examples of such excipients are parabens, thimerosal, thiomersal, chlorobutanol, bezalkonium chloride, and chelators (e.g., EDTA).

The LNP compositions of the present disclosure can be provided as a frozen liquid form or a lyophilized form. A variety of cryoprotectants may be used, including, without limitations, sucrose, trehalose, glucose, mannitol, mannose, dextrose, and the like. Once formulated with the cryoprotectant, the LNP compositions may be frozen (or lyophilized and cryopreserved) at −20° C. to −80° C. The LNP compositions may be provided to a patient in an aqueous buffered solution—thawed if previously frozen, or if previously lyophilized, reconstituted in an aqueous buffered solution at bedside. The buffered solution preferably is isotonic and suitable for e.g., intramuscular or intradermal injection. In some embodiments, the buffered solution is a phosphate-buffered saline (PBS).

### Machine Learning

To supplement protection offered by currently available quadrivalent vaccines comprising HA molecules from four standard of care influenza virus strains selected by the WHO each year, the vaccine or immunogenic compositions and methods disclosed herein further comprise one or more machine learning influenza virus HA having a molecular sequence identified or designed from a machine learning model, or one or more ribonucleic acid molecules encoding the one or more machine learning influenza virus HA, wherein the one or more machine learning influenza virus HA are selected from an H1 HA, an H3 HA, an HA from a B/Victoria lineage, an HA from a B/Yamagata lineage, or a combination thereof.

In embodiments disclosed herein, a vaccine or immunogenic composition may comprise, in addition to HAs from standard of care influenza virus strains, one or more machine learning influenza virus HA having a molecular sequence identified or designed from a machine learning model, or one or more ribonucleic acid molecules encoding the one or more machine learning influenza virus HA, as disclosed above. In certain embodiments, the one or more machine learning HA may be selected from an H1 HA, an H3 HA, an HA from a B/Victoria lineage, an HA from a B/Yamagata lineage, or a combination thereof.

The machine learning HA disclosed herein may be in any form of HA, including HA present in inactivated virus or recombinant HA, or ribonucleic acid molecules disclosed herein, including HA nucleic acid molecules (e.g., mRNA) encoding any of the aforementioned HA.

When selecting one or more machine learning influenza virus HAs, any machine learning algorithm may be used. For example, envisioned herein are any of the machine learning algorithms and methods disclosed in PCT Application Nos. WO 2021/080990 A1, entitled Systems and Methods for Designing Vaccines, WO 2021/080999 A1, entitled Systems and Methods for Predicting Biological Responses, U.S. Provisional Application No. 63/319,692, entitled Machine-Learning Techniques in Protein Design for Vaccine Generation, and U.S. Provisional Application No. 63/319,700, entitled Machine-Learning Techniques in Protein Design for Vaccine Generation, all of which are incorporated by reference in their entireties herein.

In certain embodiments, a predictive machine learning model of influenza antigenicity may be constructed, allowing prediction of antibody titer in animal models and/or humans. In certain embodiments, a machine learning model may extract feature values from input data of a training set, the features being variables deemed potentially relevant to whether or not the input data items have the associated property or properties. An ordered list of the features for the input data may be referred to as the feature vector for the input data. In certain embodiments, the machine learning model applies dimensionality reduction (e.g., via linear discrimination analysis (LDA), principle component analysis (PCA), learned deep features from a neural network, or the like) to reduce the amount of data in the feature vectors for the input data to a smaller, more representative set of data.

A set of influenza sequences to be protected against (e.g., target strains) may then be identified and a selection algorithm constructed. In certain embodiments, a system for designing vaccines is provided. The system includes one or more processors. The system includes computer storage storing executable computer instructions in which, when executed by one or more processors, cause the one or more processors to perform one or more operations. The one or more operations include applying, to a first temporal sequence data set, a plurality of driver models configured to generate output data representing one or more molecular sequences, the first temporal sequence data set indicating one or more molecular sequences and, for each of the one or more molecular sequences, one or more times of circulation for pathogenic strains including that molecular sequence as a natural antigen. The one or more operations include for each of the plurality of driver models, training the driver model by: i) receiving, from the driver model, output data representing one or more predicted molecular sequences based on the received first temporal sequence data set; ii) applying, to the output data representing the predicted one or more molecular sequences, a translational model configured to predict a biological response to molecular sequences for a plurality of translational axes to generate first translational response data representing one or more first translational responses corresponding to a particular translational axis of the plurality of translational axes based on the one or more predicted molecular sequences of the output data; iii) adjusting one or more parameters of the driver model based on the first translational response data; and iv) repeating steps i-iii for a number of iterations to generate trained translational response data representing one or more trained translational responses corresponding to the particular translational axis. The one or more operations include selecting, based on the one or more trained translational responses, a set of trained driver models of the plurality of driver models. The one or more operations include for each trained driver model of the set of trained driver models: applying, to a second temporal sequence data set, the trained driver model to generate trained output data representing one or more predicted molecular sequences for a particular season; applying, to the final output data, the translational model to generate second translational response data representing, for each translational axis of the plurality of translational axes, one or more second translational responses; and selecting, based on the second translational response data, a subset of trained driver models of the set of trained driver models.

At least one of the plurality of driver models can include a recurrent neural network. At least one of the plurality of driver models includes a long short-term memory recurrent neural network.

The output data representing one or more predicted molecular sequences based on the received first temporal sequence data set can include output data representing an antigen for each of a plurality of pathogenic seasons. The output data representing an antigen for each of a plurality of pathogenic seasons can include an antigen determined by predicting molecular sequences that will generate a maximized aggregate biological response across all pathogenic strains in circulation for a particular season. The output data representing an antigen for each of a plurality of pathogenic seasons can include an antigen determined by predicting molecular sequences that will generate a response that will effectively immunize against a maximized number of viruses in circulation for a particular season.

The plurality of translational axes can include at least one of a: ferret neutralization axis, ferret antibody forensics (AF) axis, ferret hemagglutination inhibition assay (HAI) axis, mouse neutralization axis, mouse AF axis, mouse HAI axis, human neutralization axis, human Replica AF axis, human AF axis, or human HAI axis. The number of iterations can be based on a predetermined number of iterations. The number of iterations can be based on a predetermined error value. The one or more first translational responses can include at least one of: a predicted ferret HAI titer, a predicted ferret AF titer, a predicted mouse AF titer, a predicted mouse HAI titer, a predicted human replica AF titer, a predicted human AF titer, or a predicted human HAI titer.

Selecting the set of trained driver models of the plurality of driver models can include assigning each driver model of the plurality of driver models to a class of driver models, wherein each class is associated with the particular translational axis of the plurality of translational axes used to train that driver model. Selecting the set of trained driver models of the plurality of driver models can include comparing, for each driver model of the plurality of driver models, the one or more trained translational responses of that driver model with the one or more trained translational responses of at least one other driver model assigned to the same class as that driver model.

The operations can further include for each trained driver model of the subset of trained driver models: validating that trained driver model by comparing the second translational response data corresponding to that trained driver model with observed experimental response data; and generating, in response to validating that trained driver model, a vaccine that includes the one or more molecular sequences represented by the trained output data corresponding to that trained driver model.

In an aspect, a system is provided. The system includes a computer-readable memory comprising computer-executable instructions. The system includes at least one processor configured to execute executable logic including at least one machine learning model trained to predict one or more molecular sequences, in which when the at least one processor is executing the computer-executable instructions, the at least one processor is configured to carry out one or more operations. The one or more operations include receiving temporal sequence data indicating one or more molecular sequences and, for each of the one or more molecular sequences, one or more times of circulation for pathogenic strains including that molecular sequence as a natural antigen. The one or more operations include processing the temporal sequence data through one or more data structures storing one or more portions of executable logic included in the machine learning model to predict one or more molecular sequences based on the temporal sequence data.

Predicting one or more molecular sequences based on the temporal sequence data can include predicting one or more immunological properties the predicted one or more molecular sequences will confer for use at a future time. Predicting the one or more molecular sequences based on the temporal sequence data can include predicting one or more molecular sequences that will generate a maximized aggregate biological response across all pathogenic strains of the temporal sequence data. Predicting the one or more molecular sequences based on the temporal sequence data can include predicting one or more molecular sequences that will generate a biological response that will effectively cover a maximized number of pathogenic strains of the temporal sequence data. The predicted one or more molecular sequences can be used to design a vaccine for pathogenic strains circulating during a time subsequent to the one or more times of circulation of the temporal sequence data.

The machine learning model can include a recurrent neural network.

In certain embodiments, a data processing system for predicting biological responses is provided. The system includes a computer-readable memory comprising computer-executable instructions. The system includes at least one processor configured to execute executable logic including at least one machine learning model trained to predict biological responses, wherein when the at least one processor is executing the computer-executable instructions, the at least one processor carries out one or more operations. The one or more operations include receiving first sequence data of a first molecular sequence. The one or more operations include receiving second sequence data of a second molecular sequence. The one or more operations include predicting a biological response for the second molecular sequence based at least partly on the received first and second sequence data.

The one or more operations can include receiving non-human biological response data corresponding with the first molecular sequence and the second molecular sequence. The one or more operations can include predicting the biological response is further based at least partly on the non-human biological response data. The one or more operations can include encoding the first sequence data and the second sequence data as amino acid mismatches.

The first molecular sequence can include a candidate antigen. The second molecular sequence can include a known viral strain.

Predicting the biological response can include predicting a human biological response. Predicting the biological response can include predicting at least one human biological response and at least one non-human biological response. The biological response can include an antibody titer. The machine learning model can include a deep neural network.

Machine learning techniques can be used to train a machine learning model to predict biological responses, such that incidences of false positives and false negatives are reduced. At least some of the systems and methods described can be used to, when compared with conventional techniques, efficiently process inherently sparse data, for example, by reducing the dimensionality of the data. At least some of the described systems and methods can leverage non-linear relationships in received data to increase prediction accuracy relative to traditional techniques. At least some of the described systems and methods described can be used to simultaneously predict human biological responses and non-human biological responses. At least some of the described systems and methods can be used to predict experimentally unobserved outcomes.

In certain embodiments, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method for manufacturing a vaccine by using a continuous-data algorithm. The method includes receiving a discrete-data object that may include a plurality of first discrete values, the discrete-data object may include one or more amino acid sequences. The method also includes converting the discrete-data object into a continuous-data object that may include a plurality of first continuous values. The method also includes applying, to the continuous-data object, a continuous-data algorithm to generate a continuous-result object that may include a plurality of second continuous values. The method also includes converting the continuous-result object into a discrete-result object that may include a plurality of second discrete values. The method also includes manufacturing a vaccine that may include at least one of i) a protein defined by the discrete-result object, ii) a nucleic acid capable of producing the protein defined by the discrete-result object, and a iii) delivery vehicle capable of producing the protein defined by the discrete-result object. Other embodiments of this aspect include corresponding computer systems, apparatus, and computer programs recorded on one or more computer storage devices, each configured to perform the actions of the methods.

Implementations may include one or more of the following features. The method where the one or more amino acid sequences may include: a first amino acid sequence and a second amino acid sequence, each of the first and the second amino acid sequences including respective single letters or respective letter strings. Converting the discrete-data object into the continuous-data object may include: generating, for each first discrete value, a weight-vector of weight values, each weight value representing a likelihood that the first discrete value represents a particular amino acid; generating, for each weight value of each weight-vector, a property-vector of property values, each property value representing a physiochemical property of a particular amino acid; and combining the weight-vector and the property-vector to create the first continuous values of the continuous-data object. Each weight-vector has twenty weight values, each weight value corresponding to one of twenty possible amino acids. Converting the continuous-result object into the discrete-result object may include determining, for each second continuous value, a respective single amino acid, where the determined single amino acids form the plurality of second discrete values. The method further may include: generating a plurality of candidate discrete-result objects; and excluding, from the plurality of candidate discrete-result objects, at least one discrete-result object that specifies an amino acid failing a manufacturability test. Applying the continuous-data algorithm to generate the continuous-result object may include applying a gradient descent with a loss function that determines a loss-value based on a plurality of loss criteria, the loss function may include: a first loss criteria based on an immunological response given two amino acid sequences; a second loss criteria that modifies the loss-value for sub-sequences not found in a dataset of wildtype sequences or sub-sequences not predicted to fold correctly; and a third loss criteria that, for each weight-vector, modifies the loss-value based on the greatest value in the second continuous values. Implementations of the described techniques may include hardware, a method or process, or computer software on a computer-accessible medium.

One general aspect includes a system for generating amino acid sequences, which system may include computer memory. The system may also include one or more processors. The system may also include computer-memory storing instructions that, when executed by the processors, cause the processors to perform operations that may include: receiving a discrete-data object comprising a plurality of first discrete values, the discrete-data object comprising one or more amino acid sequences; converting the discrete-data object into a continuous-data object comprising a plurality of first continuous values; applying, to the continuous-data object, a continuous-data algorithm to generate a continuous-result object comprising a plurality of second continuous values; converting the continuous-result object into a discrete-result object comprising a plurality of second discrete values; and manufacturing a vaccine comprising at least one of i) a protein defined by the discrete-result object, ii) a nucleic acid capable of producing the protein defined by the discrete-result object, and iii) a delivery vehicle capable of producing the protein defined by the discrete-result object. Other embodiments of this aspect include corresponding computer systems, apparatus, and computer programs recorded on one or more computer storage devices, each configured to perform the actions of the methods.

Implementations may include one or more of the following features. In one embodiment, there is a system where the one or more amino acid sequences may include: a first amino acid sequence and a second amino acid sequence, each of the first and the second amino acid sequences including respective single letters or respective letter strings. Converting the discrete-data object into the continuous-data object may include: generating, for each first discrete value, a weight-vector of weight values, each weight value representing a likelihood that the first discrete value represents a particular amino acid; generating, for each weight value of each weight-vector, a property-vector of property values, each property value representing a physiochemical property of a particular amino acid; and combining the weight-vector and the property-vector to create the first continuous values of the continuous-data object. Each weight-vector has twenty weight values, each weight value corresponding to one of twenty possible amino acids. Converting the continuous-result object into the discrete-result object may include determining, for each second continuous value, a respective single amino acid, where the determined single amino acids form the plurality of second discrete values. The operations further may include: generating a plurality of candidate discrete-result objects; and excluding, from the plurality of candidate discrete-result objects, at least one discrete-result object that specifies an amino acid failing a manufacturability test. Applying the continuous-data algorithm to generate the continuous-result object may include applying a gradient descent with a loss function that determines a loss-value based on a plurality of loss criteria, wherein the loss function may include: a first loss criteria based on an immunological response given two amino acid sequences; a second loss criteria that modifies the loss-value for sub-sequences not found in a dataset of wildtype sequences or sub-sequences not predicted to fold correctly; and a third loss criteria that, for each weight-vector, modifies the loss-value based on the greatest value in the second continuous values. Implementations of the described techniques may include hardware, a method or process, or computer software on a computer-accessible medium.

One general aspect includes a non-transitory, computer readable media storing instructions that, when executed by one or more processors, cause the one or more processors to perform operations that may include: receiving a discrete-data object comprising a plurality of first discrete values, the discrete-data object comprising one or more amino acid sequences; converting the discrete-data object into a continuous-data object comprising a plurality of first continuous values; applying, to the continuous-data object, a continuous-data algorithm to generate a continuous-result object comprising a plurality of second continuous values; converting the continuous-result object into a discrete-result object comprising a plurality of second discrete values; and manufacturing a vaccine comprising at least one of i) a protein defined by the discrete-result object, ii) a nucleic acid capable of producing the protein defined by the discrete-result object, and iii) a delivery vehicle capable of producing the protein defined by the discrete-result object. Other embodiments of this aspect include corresponding computer systems, apparatus, and computer programs recorded on one or more computer storage devices, each configured to perform the actions of the methods.

Implementations may include one or more of the following features. The media where the one or more amino acid sequences may include: a first amino acid sequence and a second amino acid sequence, each of the first and the second amino acid sequences including respective single letters or respective letter strings. Converting the discrete-data object into the continuous-data object may include: generating, for each first discrete value, a weight-vector of weight values, each weight value representing a likelihood that the first discrete value represents a particular amino acid; generating, for each weight value of each weight-vector, a property-vector of property values, each property value representing a physiochemical property of a particular amino acid; and combining the weight-vector and the property-vector to create the first continuous values of the continuous-data object. Each weight-vector has twenty weight values, each weight value corresponding to one of twenty possible amino acids. Converting the continuous-result object into the discrete-result object may include determining, for each second continuous value, a respective single amino acid, where the determined single amino acids form the plurality of second discrete values. Implementations of the described techniques may include hardware, a method or process, or computer software on a computer-accessible medium.

In certain embodiments, disclosed herein is an algorithm that can generate influenza antigens for use as a vaccine. In one implementation, this can include: 1) Generating a reduced-dimension space for all wildtype hemagglutinin sequences through machine learning (e.g., variational autoencoder architecture) using two steps:
a) Embedding variably into a reduced space, e.g., a model predicts mean and variance from input sequence, with embedded coordinates selected from normal distribution with predicted mean and variance; and
b) Decoding back to original sequence from reduced space location “autoencoder” loss function is then performed, reducing by the similarity of the input and output sequences.

2) Training an immune response prediction model based on location of antigen (vaccine candidate) and readout strains (target sequences) in the reduced dimensional space [input: antigen and readout embedded by model of step 1, output: measure of immune response such as antibody titer].

3) Sampling candidate vaccine component representations from the reduced space, ranking candidate vaccine component representations by predicted performance against target sequences using the model described in step 2, and identifying top candidates.

4) Decoding top candidate representations [using model from step 1b] to emit hemagglutinin sequences that may or may not have been observed in the original wildtype set.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a dimension-reducing method for generating amino acid sequences, the method being performed by a system of one or more computers. The method includes receiving one or more data objects defining a plurality of wild-type amino acid sequences. The method also includes generating, from the one or more data objects, a plurality of reduced-dimension sequences in a reduced-dimension space, where: each reduced-dimension sequence contains data respective of at least one of the wild-type amino acid sequences, the reduced-dimension space is of a lower dimensionality than the wild-type amino acid sequences, and the plurality of reduced-dimension sequences define a distribution of values along each dimension of the reduced-dimension space. The method also includes generating a plurality of candidate sequences in the reduced-dimension space using the plurality of reduced-dimension sequences. The method also includes receiving one or more data objects defining a viral amino acid sequence. The method also includes generating at least one reduced-dimension viral sequences in the reduced-dimension space. The method also includes providing, as input to a titer-predictor, each of the candidate sequences and at least one of the reduced-dimension viral sequences. The method also includes receiving, as output from the titer-predictor, a candidate-score for each of the candidate sequences. The method also includes selecting at least one candidate sequence from among the candidate sequences. The method also includes generating at least one new amino acid sequence for each of the selected candidate sequences. The method also includes providing the generated at least one amino acid sequence. The method also includes operations where each of the generated amino acid sequences is suitable for manufacturing a respective vaccine may include at least one of i) a protein defined by the generated amino acid sequence, ii) a nucleic acid capable of producing the protein defined by the generated amino acid sequence, and iii) a delivery vehicle capable of producing the protein defined by the generated amino acid sequence. Other embodiments of this aspect include corresponding computer systems, apparatus, and computer programs recorded on one or more computer storage devices, each configured to perform the actions of the methods.

Implementations may include one or more of the following features. The method includes operations where generating a plurality of reduced-dimension sequences may include creation of representations of the wild-type amino acid sequences using a variational autoencoder that predicts mean and variance values of input data. Each of the reduced-dimension sequences includes a respective group of values, and generating the plurality of candidate sequences in the reduced-dimension space may include sampling distributions of values of the plurality of reduced-dimension sequences. The titer-predictor is configured to: receive, as input, i) a first sequence in the reduced-dimension space and ii) a second sequence in the reduced-dimension space; and provide, as output, a titer-score as the candidate score, the titer-score defines a measure of biological response between the first sequence and the second sequence. Selecting the at least one candidate sequence as a selected candidate sequence may include selecting n candidate sequences with the highest candidate-scores. The method includes operations where n is a value of 1, such that a single candidate sequence is selected. The method includes operations where n is a value greater than 1, such that a plurality of candidate sequences are selected. Selecting the at least one candidate sequence as a selected candidate sequence may include selecting candidate sequences with respective candidate-scores greater than a threshold value. Each of the generated amino acid sequences is different from any of the wild-type amino acid sequences. At least one of the candidate sequences is in the plurality of reduced-dimension sequences. Implementations of the described techniques may include hardware, a method or process, or computer software on a computer-accessible medium.

One general aspect includes a system for generating amino acid sequences, the system may include computer memory. The system also includes one or more processors. The system also includes computer-memory storing instructions that, when executed by the processors, cause the processors to perform operations that may include: receiving one or more data objects defining a plurality of wild-type amino acid sequences; generating, from the one or more data objects, a plurality of reduced-dimension sequences in a reduced-dimension space, wherein: each reduced-dimension sequence contains data respective of at least one of the wild-type amino acid sequences, the reduced-dimension space is of a lower dimensionality than the wild-type amino acid sequences, and the plurality of reduced-dimension sequences define a distribution of values along each dimension of the reduced-dimension space, generating a plurality of candidate sequences in the reduced-dimension space using the plurality of reduced-dimension sequences; receiving one or more data objects defining a viral amino acid sequence; generating at least one reduced-dimension viral sequences in the reduced-dimension space; providing, as input to a titer-predictor, each of the candidate sequences and at least one of the reduced-dimension viral sequences; receiving, as output from the titer-predictor, a candidate-score for each of the candidate sequences; selecting at least one candidate sequence from among the candidate sequences; generating at least one new amino acid sequence for each of the selected candidate sequences; and providing the generated at least one amino acid sequence, wherein each of the generated amino acid sequences is suitable for manufacturing a respective vaccine comprising at least one of i) a protein defined by the generated amino acid sequence, ii) a nucleic acid capable of producing the protein defined by the generated amino acid sequence, and iii) a delivery vehicle capable of producing the protein defined by the generated amino acid sequence. Other embodiments of this aspect include corresponding computer systems, apparatus, and computer programs recorded on one or more computer storage devices, each configured to perform the actions of the methods.

Implementations may include one or more of the following features. The system where generating a plurality of reduced-dimension sequences may include creation of representations of the wild-type amino acid sequences using a variational autoencoder that predicts mean and variance values of input data. Each of the reduced-dimension sequences includes a respective group of values, and generating the plurality of candidate sequences in the reduced-dimension space may include sampling distributions of values of the plurality of reduced-dimension sequences. The titer-predictor is configured to: receive, as input, i) a first sequence in the reduced-dimension space and ii) a second sequence in the reduced-dimension space; and provide, as output, a titer-score as the candidate score, the titer-score defines a measure of biological response between the first sequence and the second sequence. Selecting the at least one candidate sequence as a selected candidate sequence may include selecting n candidate sequences with the highest candidate-scores. Implementations of the described techniques may include hardware, a method or process, or computer software on a computer-accessible medium.

One general aspect includes a non-transitory, computer readable media storing instructions that, when executed by one or more processors, cause the one or more processors to perform operations including: receiving one or more data objects defining a plurality of wild-type amino acid sequences; generating, from the one or more data objects, a plurality of reduced-dimension sequences in a reduced-dimension space, wherein: each reduced-dimension sequence contains data respective of at least one of the wild-type amino acid sequences, the reduced-dimension space is of a lower dimensionality than the wild-type amino acid sequences, and the plurality of reduced-dimension sequences define a distribution of values along each dimension of the reduced-dimension space, generating a plurality of candidate sequences in the reduced-dimension space using the plurality of reduced-dimension sequences; receiving one or more data objects defining a viral amino acid sequence; generating at least one reduced-dimension viral sequences in the reduced-dimension space; providing, as input to a titer-predictor, each of the candidate sequences and at least one of the reduced-dimension viral sequences; receiving, as output from the titer-predictor, a candidate-score for each of the candidate sequences; selecting at least one candidate sequence from among the candidate sequences; generating at least one new amino acid sequence for each of the selected candidate sequences; and providing the generated at least one amino acid sequence, wherein each of the generated amino acid sequences is suitable for manufacturing a respective vaccine comprising at least one of i) a protein defined by the generated amino acid sequence, ii) a nucleic acid capable of producing the protein defined by the generated amino acid sequence, and iii) a delivery vehicle capable of producing the protein defined by the generated amino acid sequence. Other embodiments of this aspect include corresponding computer systems, apparatus, and computer programs recorded on one or more computer storage devices, each configured to perform the actions of the methods.

Implementations may include one or more of the following features. The media where generating a plurality of reduced-dimension sequences may include creation of representations of the wild-type amino acid sequences using a variational autoencoder that predicts mean and variance values of input data. Each of the reduced-dimension sequences includes a respective group of values, and generating the plurality of candidate sequences in the reduced-dimension space may include sampling distributions of values of the plurality of reduced-dimension sequences. The titer-predictor is configured to: receive, as input, i) a first sequence in the reduced-dimension space and ii) a second sequence in the reduced-dimension space; and provide, as output, a titer-score as the candidate score, the titer-score defines a measure of biological response between the first sequence and the second sequence. Implementations of the described techniques may include hardware, a method or process, or computer software on a computer-accessible medium.

These and other aspects, features, and implementations can be expressed as methods, apparatus, systems, components, program products, methods of doing business, means or steps for performing a function, and in other ways, and will become apparent from the following descriptions, including the claims.

Implementations of the present disclosure can provide the following advantages. When compared with traditional techniques, vaccines can be designed for a future pathogenic season to confer more protection in terms of an amount of biological response for at least one pathogenic strain of that future pathogenic season. When compared with traditional techniques, vaccines can be designed for future pathogenic seasons to confer more protection in terms of breadth of effective coverage for a plurality of pathogenic strains of that future pathogenic season (that is, elicit an effective immunological response for a number of pathogenic strains in a future pathogenic season). Unlike traditional techniques, rarely observed strains that may confer “more protection” because they cross-react with more strains than frequently observed strains can be assessed and their vaccination effectiveness can be predicted.

### Methods of Measuring a Biological Response

The vaccine or immunogenic compositions disclosed herein induce biological responses (e.g., immunological response) when administered to a subject. These biological responses can be used to compare the vaccine or immunogenic compositions and determine, for example, whether the vaccine or immunogenic compositions enhance or broaden an immune response relative to vaccine or immunogenic composition without the one or more machine learning HA.

An exemplary assay that can be used to measure a biological response is a hemagglutinin inhibition assay (HAI). An HAI applies the process of hemagglutination, in which sialic acid receptors on the surface of red blood cells (RBCs) bind to a hemagglutinin glycoprotein found on the surface of an influenza virus (and several other viruses) and create a network, or lattice structure, of interconnected RBCs and virus particles, referred to as hemagglutination, which occurs in a concentration dependent manner on the virus particles. This is a physical measurement taken as a proxy as to the facility of a virus to bind to similar sialic acid receptors on pathogen-targeted cells in the body. The introduction of anti-viral antibodies raised in a human or animal immune response to another virus (which may be genetically similar or different to the virus used to bind to the RBCs in the assay) interfere with the virus-RBC interaction and change the concentration of virus sufficient to alter the concentration at which hemagglutination is observed in the assay. One goal of an HAI can be to characterize the concentration of antibodies in the antiserum or other samples containing antibodies relative to their ability to inhibit hemagglutination in the assay. The highest dilution of antibody that prevents hemagglutination is called the HAI titer (i.e., the measured response).

Another approach to measuring biological responses is to measure a potentially larger set of antibodies elicited by a human or animal immune response, which are not necessarily capable of affecting hemagglutination in the HAI assay. A common approach for this leverages enzyme-linked immunosorbent assay (ELISA) techniques, in which a viral antigen (e.g., hemagglutinin) is immobilized to a solid surface, and then antibodies from the antisera are allowed to bind to the antigen. The readout measures the catalysis of a substrate of an exogenous enzyme complexed to either the antibodies from the antisera, or to other antibodies which themselves bind to the antibodies of the antisera. Catalysis of the substrate gives rise to easily detectable products. There are many variations of this sort of in vitro assay. One such variation is called antibody forensics (AF), which is a multiplexed bead array technique that allowed a single sample of serum to be measured against many antigens simultaneously. These measurements characterize the concentration and total antibody recognition, as compared to HAI titers, which are taken to be more specifically related to interference with sialic acid binding by hemagglutinin molecules. Therefore, an antisera's antibodies may in some cases have proportionally higher or lower measurements than the corresponding HAI titer for one virus's hemagglutinin molecules relative to another virus's hemagglutinin molecules; in other words, these two measurements, AF and HAI, are not generally linearly related.

Another method of measuring humoral immune response includes a viral neutralization assay (e.g., microneutralization assay), wherein an antibody titer is measured by a reduction in plaques, foci, and/or fluorescent signal, depending on the specific neutralization assay technique, in permissive cultured cells following incubation of virus with serial dilutions of an antibody/serum sample.

### Vaccine or Immunogenic Compositions

The present disclosure provides a multivalent vaccine or immunogenic composition comprising influenza virus HAs from the standard of care influenza virus strains (e.g., HAs from at least three or at least four standard of care influenza strains) or ribonucleic acid molecules encoding the influenza virus HAs from standard of care influenza strains, and one or more influenza virus HA having a molecular sequence identified or designed from a machine learning model or one or more ribonucleic acid molecules encoding the one or more machine learning influenza virus HA.

In certain aspects, disclosed herein is a vaccine or immunogenic composition comprising:
(a) a first influenza virus hemagglutinin (HA) wherein the first influenza virus HA is an H1 HA from a first standard of care influenza virus strain, or a first ribonucleic acid molecule encoding the first influenza virus H1 HA;
(b) a second influenza virus HA wherein the second influenza virus HA is an H3 HA from a second standard of care influenza virus strain, or a second ribonucleic acid molecule encoding the second influenza virus H3 HA;
(c) a third influenza virus HA wherein the third influenza virus HA is from a third standard of care influenza virus strain from the B/Victoria lineage, or a third ribonucleic acid molecule encoding the third influenza virus HA from the B/Victoria lineage;
(d) a fourth influenza virus HA wherein the fourth influenza virus HA is from a fourth standard of care influenza virus strain from the B/Yamagata lineage, or a fourth ribonucleic acid molecule encoding the fourth influenza virus HA from the B/Yamagata lineage; and
(e) one or more machine learning influenza virus HA having a molecular sequence identified or designed from a machine learning model, or one or more ribonucleic acid molecules encoding the one or more machine learning influenza virus HA, wherein the one or more machine learning influenza virus HA are selected from an H1 HA, an H3 HA, an HA from a B/Victoria lineage, an HA from a B/Yamagata lineage, or a combination thereof.

In certain embodiments of the vaccine or immunogenic composition disclosed herein, the one or more machine learning influenza virus HAs comprise a fifth influenza virus HA, wherein the fifth influenza virus HA is an H3 HA, and wherein the fifth influenza H3 HA is antigenically dissimilar than the second influenza H3 HA. In certain embodiments, the fifth influenza H3 HA is antigenically similar to the second influenza H3 HA. In certain embodiments, the fifth influenza H3 HA enhances or broadens a protective immune response induced by the second influenza H3 HA. In certain embodiments, the fifth influenza H3 HA is from a different clade than the second influenza H3 HA, and in certain embodiments, the fifth influenza H3 HA is from the same clade as the second influenza H3 HA. In certain embodiments, the fifth H3 HA is from the 3C.2A clade, and in certain embodiments, the fifth H3 HA is from the 3C.3A clade. In certain embodiments, the one or more machine learning influenza virus HAs comprise two or more H3 HAs, such as 2, 3, or 4 H3 HAs.

In certain additional embodiments of the vaccine or immunogenic composition disclosed herein, the one or more machine learning influenza virus HAs is a fifth influenza virus HA, wherein the fifth influenza virus HA is an H1 HA, and wherein the fifth influenza H1 HA is antigenically dissimilar than the first influenza H1 HA. In certain embodiments, the fifth influenza H1 HA is antigenically similar to the first influenza H1 HA. In certain embodiments, the fifth influenza H1 HA enhances or broadens a protective immune response induced by the first influenza H1 HA. In certain embodiments, the fifth influenza H1 HA is from a different clade than the first influenza H1 HA, and in certain embodiments, the fifth H1 HA is from the same clade as the first influenza H1 HA. In certain embodiments, the H1 HA is from the 6B.1 clade, and in certain embodiments, the H1 HA is from the 6B.1A subclade. In certain embodiments, the one or more machine learning influenza virus HAs comprise two or more H1 HAs, such as 2, 3, or 4 H1 HAs.

In certain additional embodiments of the vaccine or immunogenic composition disclosed herein, the one or more machine learning influenza virus HAs is a fifth influenza virus HA, from a B/Victoria lineage, and wherein the fifth influenza is antigenically dissimilar than the third influenza virus HA. In certain embodiments, the fifth influenza virus HA is antigenically similar to the third influenza virus HA. In certain embodiments, the fifth influenza virus HA enhances or broadens a protective immune response induced by the third influenza virus HA. In certain embodiments, the fifth influenza virus HA is from a different clade than the third influenza virus HA, and in certain embodiments, the fifth influenza virus HA is from the same clade as the third influenza virus HA. In certain embodiments, the fifth influenza virus HA is from the V1A clade of B/Victoria, and in certain embodiments, the fifth influenza virus HA is from the V1A.1 subclade, V1A.2 subclade, or the V1A.3 subclade of B/Victoria. In certain embodiments, the one or more machine learning influenza virus HAs comprise two or more HAs from a B/Victoria lineage, such as 2, 3, or 4 HAs from a B/Victoria lineage.

In certain additional embodiments of the vaccine or immunogenic composition disclosed herein, the one or more machine learning influenza virus HAs is a fifth influenza virus HA from a B/Yamagata lineage, and wherein the fifth influenza is antigenically dissimilar than the fourth influenza virus HA. In certain embodiments, the fifth influenza virus HA is antigenically similar to the fourth influenza virus HA. In certain embodiments, the fifth influenza virus HA enhances or broadens a protective immune response induced by the fourth influenza virus HA. In certain embodiments, the fifth influenza virus HA is from a different clade than the fourth influenza virus HA, and in certain embodiments, the fifth influenza virus HA is from the same clade as the fourth influenza virus HA. In certain embodiments, the fifth influenza virus HA is from the Y1 clade of B/Yamagata, and in certain embodiments, the fifth influenza virus HA is from the Y2 clade of B/Yamagata. In certain embodiments, the fifth influenza virus HA is from the Y3 clade of B/Yamagata. In certain embodiments, the one or more machine learning influenza virus HAs comprise two or more HAs from a B/Yamagata lineage, such as 2, 3, or 4 HAs from a B/Yamagata lineage.

In certain aspects of the disclosure, the vaccine or immunogenic composition comprises a sixth influenza virus HA, wherein the sixth influenza virus HA is an H1 HA or an H3 HA having a molecular sequence identified or designed from a machine learning model, or nucleic acid molecule encoding the sixth influenza virus HA.

In certain embodiments, the sixth influenza is an H1 HA and is antigenically dissimilar than the first influenza H1 HA, enhances or broadens a protective immune response induced by the first influenza H1 HA, is from a different clade than the first influenza H1 HA, is from a same clade as the first influenza H1 HA, or is antigenically similar to the first influenza H1 HA. In certain embodiments, the sixth influenza is an H3 HA and is antigenically dissimilar than the second influenza H3 HA, enhances or broadens a protective immune response induced by the second influenza H3 HA, is from a different clade than the second influenza H3 HA, is from a same clade as the second influenza H3 HA, or is antigenically similar to the second influenza H3 HA.

In certain embodiments of the vaccine or immunogenic compositions disclosed herein, the first influenza virus HA is an H1 HA from an H1N1 influenza virus strain and the second influenza virus HA is an H3 HA from an H3N2 influenza virus strain.

One or more of the HA in the multivalent vaccine or immunogenic composition may be recombinant HA and can be formulated and packaged, alone or in combination with other recombinant HA antigens, including in combination with HA from standard of care influenza virus strains and/or machine learning HA. In certain embodiments, the recombinant HA is formulated with one, two, or three additional recombinant HA antigens, such as one, two, or three additional recombinant antigens from standard of care influenza virus strains. In certain embodiments, the recombinant HA is formulated with three additional recombinant HA antigens to produce a quadrivalent vaccine or immunogenic composition. In certain embodiments, the vaccine or immunogenic composition may contain four recombinant antigens from standard of care influenza virus strains and one or more, such as one, two, three, or four machine learning influenza virus HA.

In certain embodiments, the vaccine or immunogenic composition may comprise a recombinant H3 HA, a recombinant H1 HA, a recombinant HA from a B/Victoria lineage, a recombinant HA from a B/Yamagata lineage, and a recombinant machine learning H3 HA.

In certain embodiments, the vaccine or immunogenic composition may comprise a recombinant H3 HA, a recombinant H1 HA, a recombinant HA from a B/Victoria lineage, a recombinant HA from a B/Yamagata lineage, and a recombinant machine learning H1 HA.

In certain embodiments, the vaccine or immunogenic composition may comprise a recombinant H3 HA, a recombinant H1 HA, a recombinant HA from a B/Victoria lineage, a recombinant HA from a B/Yamagata lineage, a recombinant machine learning H3 HA, and a recombinant machine learning H1 HA.

In certain embodiments, the vaccine or immunogenic composition may comprise a recombinant H3 HA, a recombinant H1 HA, a recombinant HA from a B/Victoria lineage, a recombinant HA from a B/Yamagata lineage, a recombinant machine learning H3 HA, a recombinant machine learning H1 HA, and a recombinant machine learning HA from a B/Victoria lineage.

In certain embodiments, the vaccine or immunogenic composition may comprise a recombinant H3 HA, a recombinant H1 HA, a recombinant HA from a B/Victoria lineage, a recombinant HA from a B/Yamagata lineage, a recombinant machine learning H3 HA, a recombinant machine learning H1 HA, and a recombinant machine learning HA from a B/Yamagata lineage.

In certain embodiments, the vaccine or immunogenic composition may comprise a recombinant H3 HA, a recombinant H1 HA, a recombinant HA from a B/Victoria lineage, a recombinant HA from a B/Yamagata lineage, a recombinant machine learning H3 HA, a recombinant machine learning H1 HA, a recombinant machine learning HA from a B/Victoria lineage, and a recombinant machine learning HA from a B/Yamagata lineage.

In any of the embodiments where the vaccine or immunogenic composition comprises recombinant HA, one or more of the recombinant HA in the vaccine or immunogenic composition can be replaced by one or more HA present in an inactivated influenza virus or by one or more ribonucleic acid molecules encoding the influenza virus HA. For example, in certain embodiments, the vaccine or immunogenic composition may comprise an inactivated influenza virus H3 HA, an inactivated influenza virus H1 HA, an inactivated influenza virus HA from a B/Victoria lineage, an inactivated influenza virus HA from a B/Yamagata lineage, and a recombinant machine learning H3 HA or a ribonucleic acid encoding a machine learning influenza virus H3 HA. In certain embodiments, the vaccine or immunogenic composition may comprise a ribonucleic acid encoding an influenza virus H3 HA, a ribonucleic acid encoding an influenza virus H1 HA, a ribonucleic acid encoding an influenza virus HA from a B/Victoria lineage, a ribonucleic acid encoding an influenza virus HA from a B/Yamagata lineage, and a recombinant machine learning H3 HA or a ribonucleic acid encoding a machine learning influenza virus H3 HA.

One or more of the HA in the multivalent vaccine or immunogenic composition may be present in an inactivated influenza virus, as disclosed herein, and can be formulated and packaged, alone or in combination with other HA, including in combination with HA from standard of care influenza virus strains and/or machine learning HA, including recombinant HA, other HA present in an inactivated influenza virus, or a ribonucleic acid encoding the HA.

In certain embodiments, the HA present in an inactivated influenza virus is formulated with one, two, or three additional HAs present in an inactivated influenza virus, such as one, two, or three additional HAs from standard of care influenza virus strains. In certain embodiments, the HA present in an inactivated influenza virus is formulated with three additional HAs present in an inactivated influenza virus to produce a quadrivalent vaccine or immunogenic composition. In certain embodiments, the vaccine or immunogenic composition may contain four HAs present in an inactivated influenza virus from standard of care influenza virus strains and one or more, such as one, two, three, or four machine learning influenza virus HA.

In certain embodiments, the vaccine or immunogenic composition may comprise a H3 HA, a H1 HA, a HA from a B/Victoria lineage, a HA from a B/Yamagata lineage, and a machine learning H3 HA, wherein each HA in the composition is present in an inactivated influenza virus.

In certain embodiments, the vaccine or immunogenic composition may comprise a H3 HA, a H1 HA, a HA from a B/Victoria lineage, a HA from a B/Yamagata lineage, and a machine learning H1 HA, wherein each HA in the composition is present in an inactivated influenza virus.

In certain embodiments, the vaccine or immunogenic composition may comprise a H3 HA, a H1 HA, a HA from a B/Victoria lineage, a HA from a B/Yamagata lineage, a machine learning H3 HA, and a machine learning H1 HA, wherein each HA in the composition is present in an inactivated influenza virus.

In certain embodiments, the vaccine or immunogenic composition may comprise a H3 HA, a H1 HA, a HA from a B/Victoria lineage, a HA from a B/Yamagata lineage, a machine learning H3 HA, a machine learning H1 HA, and a machine learning HA from a B/Victoria lineage, wherein each HA in the composition is present in an inactivated influenza virus.

In certain embodiments, the vaccine or immunogenic composition may comprise a H3 HA, a H1 HA, a HA antigen from a B/Victoria lineage, a HA from a B/Yamagata lineage, a machine learning H3 HA, a machine learning H1 HA, and a machine learning HA from a B/Yamagata lineage, wherein each HA in the composition is present in an inactivated influenza virus.

In certain embodiments, the vaccine or immunogenic composition may comprise a H3 HA, a H1 HA, a HA from a B/Victoria lineage, a HA from a B/Yamagata lineage, a machine learning H3 HA, a machine learning H1 HA, a machine learning HA from a B/Victoria lineage, and a machine learning HA from a B/Yamagata lineage, wherein each HA in the composition is present in an inactivated influenza virus.

In any of the embodiments where the vaccine or immunogenic composition comprises an HA present in an inactivated influenza virus, one more of the HA present in an inactivated influenza virus can be replaced by one or more recombinant HA or by one or more ribonucleic acid molecules encoding the influenza virus HA.

Each recombinant HA may be present in the compositions disclosed herein in an amount effected to induce an immune response in a subject to which the composition is administered. In certain embodiments, each recombinant HA may be present in the vaccine or immunogenic compositions disclosed herein in an amount ranging, for example, from about 5 μg to about 120 μg, such as from about 10 μg to about 60 μg, or about 15 μg to about 45 μg. In certain embodiments, each recombinant HA is present in the vaccine or immunogenic composition disclosed herein an amount of about 5 μg, about 10 μg, about 15 μg, about 20 μg, about 25 μg, about 30 μg, about 35 μg, about 40 μg, about 45 μg, about 50 μg, about 55 μg, or about 60 μg.

Ribonucleic acid molecules encoding an HA may each be present in the vaccine or immunogenic compositions disclosed herein in an amount ranging, for example, from about 5 μg to about 120 μg, such as from about 10 μg to about 60 μg, or about 15 μg to about 45 μg. In certain embodiments, ribonucleic acid molecules encoding an HA are present in the vaccine or immunogenic composition disclosed herein an amount of about 5 μg, about 10 μg, about 15 μg, about 20 μg, about 25 μg, about 30 μg, about 35 μg, about 40 μg, about 45 μg, about 50 μg. about 55 μg, about 60 μg, about 65 μg, about 70 μg, about 75 μg, about 80 μg, about 85 μg, about 90 μg, about 95 μg, or about 100 μg.

Each HA present in an inactivated virus may be present in the compositions disclosed herein in an amount effective to induce an immune response in a subject to which the composition is administered. In certain embodiments, each HA present in an inactivated virus may be present in the vaccine or immunogenic compositions disclosed herein in an amount ranging, for example, from about 5 μg to about 120 μg, such as from about 10 μg to about 100 μg, about 10 μg to about 60 μg, or about 15 μg to about 45 μg. In certain embodiments, each HA present in an inactivated virus is present in the vaccine or immunogenic composition disclosed herein an amount of about 5 μg, about 10 μg. about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg.

Further disclosed herein is a vaccine or immunogenic composition, comprising:
(a) a first influenza virus HA wherein the first influenza virus HA is an H1 HA from a first standard of care influenza virus strain, or a first ribonucleic acid molecule encoding the first influenza virus H1 HA;
(b) a second influenza virus HA wherein the second influenza virus HA is from a second standard of care influenza virus strain from the B/Victoria lineage, or a second ribonucleic acid molecule encoding the second influenza virus HA from the B/Victoria lineage;
(c) a third influenza virus HA wherein the third influenza virus HA is from a third standard of care influenza virus strain from the B/Yamagata lineage, or a third ribonucleic acid molecule encoding the third influenza virus HA from the B/Yamagata lineage; and
(d) a fourth influenza virus HA, wherein the fourth influenza virus HA is a machine learning influenza virus H3 HA having a molecular sequence identified or designed from a machine learning model, or one or more ribonucleic acid molecules encoding the machine learning influenza virus H3 HA.

Further disclosed herein is a vaccine or immunogenic composition, comprising:
(a) a first influenza virus HA wherein the first influenza virus HA is an H3 HA from a first standard of care influenza virus strain, or a first ribonucleic acid molecule encoding the first influenza virus H3 HA;
(b) a second influenza virus HA wherein the second influenza virus HA is from a second standard of care influenza virus strain from the B/Victoria lineage, or a second ribonucleic acid molecule encoding the second influenza virus HA from the B/Victoria lineage;
(c) a third influenza virus HA wherein the third influenza virus HA is from a third standard of care influenza virus strain from the B/Yamagata lineage, or a third ribonucleic acid molecule encoding the third influenza virus HA from the B/Yamagata lineage; and
(d) a fourth influenza virus HA, wherein the fourth influenza virus HA is a machine learning influenza virus H1 HA having a molecular sequence identified or designed from a machine learning model, or one or more ribonucleic acid molecules encoding the machine learning influenza virus H1 HA.

In certain embodiments, the vaccine or immunogenic compositions disclosed herein further comprise an additional influenza virus H1 HA, and additional influenza virus H3 HA, an influenza virus HA from the B/Victoria lineage, and/or an influenza virus HA from the B/Yamagata lineage, wherein each additional HA has a molecular sequence identified or designed from a machine learning model, or a ribonucleic acid molecule encoding the additional machine learning influenza virus HA.

In certain embodiments, the vaccine or immunogenic composition is a quadrivalent HA vaccine. In certain embodiments, the vaccine or immunogenic composition is a pentavalent HA vaccine. In certain embodiments, the vaccine or immunogenic composition is a hexavalent HA vaccine. In certain embodiments, the vaccine or immunogenic composition is a heptavalent HA vaccine. In certain embodiments, the vaccine or immunogenic composition is an octavalent HA vaccine. In certain embodiments, the vaccine or immunogenic composition is a multivalent vaccine or immunogenic comprising more than 8 different HA molecules.

The vaccine or immunogenic composition can also further comprise an adjuvant. Adjuvants can include a suspension of minerals (alum, aluminum salts, including, for example, aluminum hydroxide/oxyhydroxide (AlOOH), aluminum phosphate (AlPO₄), aluminum hydroxyphosphate sulfate (AAHS) and/or potassium aluminum sulfate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as lipids and costimulatory molecules. Exemplary biological adjuvants include AS04 (Didierlaurent, A. M. et al, J. Immunol., 2009, 183: 6186-6197), IL-2, RANTES, GM-CSF, TNF-α, IFN-γ, G-CSF, LFA-3, CD72, B7-1, B7-2, OX-40L and 41 BBL.

In certain embodiments, the adjuvant is a squalene-based adjuvant comprising an oil-in-water adjuvant emulsion comprising at least: squalene, an aqueous solvent, a polyoxyethylene alkyl ether hydrophilic nonionic surfactant, and a hydrophobic nonionic surfactant. In certain embodiments, the emulsion is thermoreversible, optionally wherein 90% of the population by volume of the oil drops has a size less than 200 nm.

In certain embodiments, the polyoxyethylene alkyl ether is of formula CH₃—(CH₂)ₓ—(O—CH₂—CH₂)ₙ—OH, in which n is an integer from 10 to 60, and x is an integer from 11 to 17. In certain embodiments, the polyoxyethylene alkyl ether surfactant is polyoxyethylene(12) cetostearyl ether.

In certain embodiments, 90% of the population by volume of the oil drops has a size less than 160 nm. In certain embodiments, 90% of the population by volume of the oil drops has a size less than 150 nm. In certain embodiments, 50% of the population by volume of the oil drops has a size less than 100 nm. In certain embodiments, 50% of the population by volume of the oil drops has a size less than 90 nm.

In certain embodiments, the adjuvant further comprises at least one alditol, including, but not limited to, glycerol, erythritol, xylitol, sorbitol and mannitol.

In certain embodiments the hydrophilic/lipophilic balance (HLB) of the hydrophilic nonionic surfactant is greater than or equal to 10. In certain embodiments, the HLB of the hydrophobic nonionic surfactant is less than 9. In certain embodiments, the HLB of the hydrophilic nonionic surfactant is greater than or equal to 10 and the HLB of the hydrophobic nonionic surfactant is less than 9.

In certain embodiments, the hydrophobic nonionic surfactant is a sorbitan ester, such as sorbitan monooleate, or a mannide ester surfactant. In certain embodiments, the amount of squalene is between 5 and 45%. In certain embodiments, the amount of polyoxyethylene alkyl ether surfactant is between 0.9 and 9%. In certain embodiments, the amount of hydrophobic nonionic surfactant is between 0.7 and 7%. In certain embodiments, the adjuvant comprises: i) 32.5% of squalene, ii) 6.18% of polyoxyethylene(12) cetostearyl ether, iii) 4.82% of sorbitan monooleate, and iv) 6% of mannitol.

In certain embodiments, the adjuvant further comprises an alkylpolyglycoside and/or a cryoprotective agent, such as a sugar, in particular dodecylmaltoside and/or sucrose.

97| In certain embodiments, the adjuvant comprises AF03, as described in Klucker et al., J. Pharm. Sci. 2012, 101(12):4490-500, which is hereby incorporated by reference in its entirety. In certain embodiments, the adjuvant comprises a liposome-based adjuvant, such as SPA14, as described for example in WO 2022/090359, which is hereby incorporated by reference in its entirety.

In addition to the HAs and optional adjuvant, the vaccine or immunogenic composition may also further comprise one or more pharmaceutically acceptable excipients. In general, the nature of the excipient will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, vaccine or immunogenic compositions to be administered can contain minor amounts of non-toxic auxiliary substances, such as wetting or emulsifying agents, pharmaceutically acceptable salts to adjust the osmotic pressure, preservatives, stabilizers, buffers, sugars, amino acids, and pH buffering agents and the like, for example sodium acetate or sorbitan monolaurate.

Typically, the vaccine or immunogenic composition is a sterile, liquid solution formulated for parenteral administration, such as intravenous, subcutaneous, intraperitoneal, intradermal, or intramuscular. The vaccine or immunogenic composition may also be formulated for intranasal or inhalation administration. The vaccine or immunogenic composition can also be formulated for any other intended route of administration.

In some embodiments, a vaccine or immunogenic composition is formulated for intradermal injection, intranasal administration, or intramuscular injection. In some embodiments, injectables are prepared in conventional forms, either as liquid solutions or suspensions, solid forms suitable for solution or suspension in liquid prior to injection, or as emulsions. In some embodiments, injection solutions and suspensions are prepared from sterile powders or granules. General considerations in the formulation and manufacture of pharmaceutical agents for administration by these routes may be found, for example, in Remington's Pharmaceutical Sciences, 19^{th} ed., Mack Publishing Co., Easton, PA, 1995; incorporated herein by reference. At present the oral or nasal spray or aerosol route (e.g., by inhalation) are most commonly used to deliver therapeutic agents directly to the lungs and respiratory system. In some embodiments, the vaccine or immunogenic composition is administered using a device that delivers a metered dosage of the vaccine or immunogenic composition. Suitable devices for use in delivering intradermal pharmaceutical compositions described herein include short needle devices such as those described in U.S. Pat. Nos. 4,886,499, 5,190,521, 5,328,483, 5,527,288, 4,270,537, 5,015,235, 5,141,496, 5,417,662 (all of which are incorporated herein by reference). Intradermal compositions may also be administered by devices which limit the effective penetration length of a needle into the skin, such as those described in WO1999/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Patent No. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO1997/37705, and WO1997/13537 (all of which are incorporated herein by reference). Also suitable are ballistic powder/particle delivery devices which use compressed gas to accelerate vaccine in powder form through the outer layers of the skin to the dermis. Additionally, conventional syringes may be used in the classical mantoux method of intradermal administration.

Preparations for parenteral administration typically include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

### Kits

Further disclosed herein are kits for the vaccine or immunogenic compositions disclosed herein. Kits may include a suitable container comprising the vaccine or immunogenic composition or a plurality of containers comprising different components of the vaccine or immunogenic composition, optionally with instructions for use.

In certain embodiments, the kit may comprise a plurality of containers, including, for example, a first container comprising (a) a first influenza virus HA wherein the first influenza virus HA is an H1 HA from a first standard of care influenza virus strain; (b) a second influenza virus HA wherein the second influenza virus HA is an H3 HA from a second standard of care influenza virus strain; (c) a third influenza virus HA wherein the third influenza virus HA is from a third standard of care influenza virus strain from the B/Victoria lineage; and (d) a fourth influenza virus HA wherein the fourth influenza virus HA is from a fourth standard of care influenza virus strain from the B/Yamagata lineage; and a second container comprising one or more machine learning influenza virus HA having a molecular sequence identified or designed from a machine learning model, or one or more ribonucleic acid molecules encoding the one or more machine learning influenza virus HA, wherein the one or more machine learning influenza virus HA are selected from an H1 HA, an H3 HA, an HA from a B/Victoria lineage, an HA from a B/Yamagata lineage, or a combination thereof.

In certain embodiments, each of the first, second, third, and fourth influenza virus HA in the first container is a recombinant influenza virus HA and the one or more machine learning influenza virus HA in the second container is a recombinant influenza virus HA. Alternatively, the one or more machine learning influenza virus HA in the second container is present in an inactivated virus or the second container comprises one or more ribonucleic acid molecules encoding the one or more machine learning influenza virus HA.

In certain embodiments, each of the first, second, third, and fourth influenza virus HA in the first container is present in an inactivated influenza virus and the one or more machine learning influenza virus HA in the second container is present in an inactivated virus. Alternatively, the one or more machine learning influenza virus HA in the second container is a recombinant HA or the second container comprises one or more ribonucleic acid molecules encoding the one or more machine learning influenza virus HA.

In certain embodiments, each of the first, second, third, and fourth influenza virus HA in the first container is present as ribonucleic acid molecules each encoding the respective influenza virus HA and the one or more machine learning influenza virus HA in the second container is present as ribonucleic acid molecules each encoding the respective influenza virus HA. Alternatively, the one or more machine learning influenza virus HA in the second container is a recombinant HA or is present in an inactivated influenza virus.

### Nucleic Acids, Cloning, and Expression Systems

The present disclosure further provides nucleic acid molecules encoding the disclosed HAs. The nucleic acids may be used, for example, to express recombinant HA, which can be used in a vaccine or immunogenic composition or as a component of the vaccine or immunogenic composition. The nucleic acids may comprise DNA or RNA and may be wholly or partially synthetic or recombinant. Reference to a nucleotide sequence as set out herein encompasses a DNA molecule with the specified sequence and encompasses an RNA molecule with the specified sequence in which U is substituted for T, or a derivative thereof, such as pseudouridine, unless context requires otherwise. Other nucleotide derivatives or modified nucleotides can be incorporated into the nucleic acid molecules encoding the disclosed HAs.

The present disclosure also provides constructs in the form of a vector (e.g., plasmids, phagemids, cosmids, transcription or expression cassettes, artificial chromosomes, etc.) comprising an artificial nucleic acid molecule encoding a HA as disclosed herein. The disclosure further provides a host cell which comprises one or more constructs as above.

Also provided are methods of making the HA encoded by these nucleic acid molecules. The HA polypeptides may be produced using recombinant techniques. The production and expression of recombinant proteins is well known in the art and can be carried out using conventional procedures, such as those disclosed in Sambrook et al., Molecular Cloning: A Laboratory Manual (4th Ed. 2012), Cold Spring Harbor Press. For example, expression of the HA polypeptide may be achieved by culturing under appropriate conditions host cells containing the nucleic acid molecule encoding the HA as disclosed herein. Following production by expression, the HA may be isolated and/or purified using any suitable technique, then used as appropriate.

Systems for cloning and expression of a polypeptide in a variety of different host cells are well known in the art. Any protein expression system (e.g., stable or transient) compatible with the constructs disclosed in this application may be used to produce the HAs described herein.

Suitable vectors can be chosen or constructed, so that they contain appropriate regulatory sequences, including promoter sequences, terminator sequences, polyadenylation sequences, enhancer sequences, marker genes and other sequences as appropriate.

For expressing recombinant HA, nucleic acids encoding HA can be introduced into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. These techniques are well known in the art. (See, e.g., “Current Protocols in Molecular Biology,” Ausubel et al. eds., John Wiley & Sons, 2010). DNA introduction may be followed by a selection method (e.g., antibiotic resistance) to select cells that contain the vector.

The host cell may be a plant cell, a yeast cell, or an animal cell. Animal cells encompass invertebrate (e.g., insect cells), non-mammalian vertebrate (e.g., avian, reptile and amphibian) and mammalian cells. In one embodiment, the host cell is a mammalian cell. Examples of mammalian cells include, but are not limited to COS-7 cells, HEK293 cells; baby hamster kidney (BHK) cells; Chinese hamster ovary (CHO) cells; mouse sertoli cells; African green monkey kidney cells (VERO-76); human cervical carcinoma cells (e.g., HeLa); canine kidney cells (e.g., MDCK), and the like. In one embodiment, the host cells are insect cells.

### Methods of Use

The present disclosure provides methods of administering the vaccine or immunogenic compositions described herein to a subject. The methods may be used to vaccinate a subject against an influenza virus. In some embodiments, the vaccination method comprises administering to a subject in need thereof a vaccine or immunogenic composition comprising the HAs and/or ribonucleic acid molecules as described herein and an optional adjuvant in an amount effective to vaccinate the subject against influenza virus. Likewise, the present disclosure provides a vaccine or immunogenic composition comprising the HAs and/or ribonucleic acid molecules as described herein, for use in vaccinating a subject against influenza virus. The present disclosure also provides the use of a vaccine or immunogenic composition comprising the HAs and/or ribonucleic acid molecules as described herein for the manufacture of a medicament for vaccinating a subject against influenza virus.

The present disclosure also provides methods of immunizing a subject against influenza virus, comprising administering to the subject an immunologically effective amount of a vaccine or immunogenic composition comprising the HAs and/or ribonucleic acid molecules as described herein and an optional adjuvant. Likewise, the present disclosure provides a vaccine or immunogenic composition comprising the HAs and/or ribonucleic acid molecules as described herein, for use in immunizing a subject against influenza virus. The present disclosure also provides the use of a vaccine or immunogenic composition comprising the HAs and/or ribonucleic acid molecules as described herein for the manufacture of a medicament for immunizing a subject against influenza virus.

In some embodiments, the method or use prevents influenza virus infection or disease in the subject. In some embodiments, the method or use raises a protective immune response in the subject. In some embodiments, the protective immune response is an antibody response.

The methods of immunizing (or related uses) provided herein can elicit a broadly neutralizing immune response against one or more influenza viruses. Accordingly, in various embodiments, the composition described herein can offer broad cross-protection against different types of influenza viruses. In some embodiments, the composition offers cross-protection against avian, swine, seasonal, and/or pandemic influenza viruses. In some embodiments, the methods of immunizing (or related uses) are capable of eliciting an improved immune response against one or more seasonal influenza strains (e.g., a standard of care strain). For example, the improved immune response may be an improved humoral immune response. In some embodiments, the methods of immunizing (or related uses) are capable of eliciting an improved immune response against one or more pandemic influenza strains. In some embodiments, the methods of immunizing (or related uses) are capable of eliciting an improved immune response against one or more swine influenza strains. In some embodiments, the methods of immunizing (or related uses) are capable of eliciting an improved immune response against one or more avian influenza strains.

Also provided are methods of preventing influenza virus disease in a subject, comprising administering to the subject a vaccine or immunogenic composition comprising the HAs and/or ribonucleic acid molecules as described herein and an optional adjuvant in an amount effective to prevent influenza virus disease in the subject. Likewise, the present disclosure provides a vaccine or immunogenic composition comprising the HAs and/or ribonucleic acid molecules as described herein and an optional adjuvant, for use in preventing influenza virus disease in a subject. The present disclosure also provides the use of a vaccine or immunogenic composition comprising the HAs and/or ribonucleic acid molecules as described herein and an optional adjuvant for the manufacture of a medicament for preventing influenza virus disease in a subject.

Also provided are methods of inducing an immune response against an influenza virus HA in a subject, comprising administering to the subject a vaccine or immunogenic composition comprising the HAs and/or ribonucleic acid molecules as described herein and an optional adjuvant. Likewise, the present disclosure provides a vaccine or immunogenic composition comprising the HAs and/or ribonucleic acid molecules as described herein and an optional adjuvant, for use in inducing an immune response against an influenza virus HA in a subject. The present disclosure also provides the use of a vaccine or immunogenic composition comprising the HAs and/or ribonucleic acid molecules as described herein and an optional adjuvant for the manufacture of a medicament for use in inducing an immune response against an influenza virus in a subject.

Vaccine or immunogenic compositions comprising the HAs and/or ribonucleic acid molecules as described herein and an optional adjuvant may be administered prior to or after development of one or more symptoms of an influenza infection. That is, in some embodiments, the vaccine or immunogenic compositions described herein may be administered prophylactically to prevent influenza infection or ameliorate the symptoms of a potential influenza infection. In some embodiments, a subject is at risk of influenza virus infection if the subject will be in contact with other individuals or livestock (e.g., swine) known or suspected to have been infected with pandemic influenza virus and/or if the subject will be present in a location in which influenza infection is known or thought to be prevalent or endemic. In some embodiments, the vaccine or immunogenic compositions are administered to a subject suffering from an influenza infection, or the subject is displaying one or more symptoms commonly associated with influenza infection. In some embodiments, the subject is known or believed to have been exposed to an influenza virus. In some embodiments, a subject is at risk or susceptible to an influenza infection if the subject is known or believed to have been exposed to the influenza virus. In some embodiments, a subject is known or believed to have been exposed to the influenza virus if the subject has been in contact with other individuals or livestock (e.g., swine) known or suspected to have been infected with pandemic influenza virus and/or if the subject is or has been present in a location in which influenza infection is known or thought to be prevalent or endemic. The vaccine or immunogenic compositions disclosed herein may be used to treat or prevent disease caused by either or both a seasonal or a pandemic influenza strain.

Vaccine or immunogenic compositions in accordance with the disclosure may be administered in any amount or dose appropriate to achieve a desired outcome. In some embodiments, the desired outcome is induction of a lasting adaptive immune response against a broad spectrum of influenza strains, including both seasonal and pandemic strains. In some embodiments, the desired outcome is reduction in intensity, severity, and/or frequency, and/or delay of onset of one or more symptoms of influenza infection. The dose required may vary from subject to subject depending on the species, age, weight and general condition of the subject, the severity of the infection being treated, the particular composition being used and its mode of administration.

In various embodiments, the vaccine or immunogenic compositions described herein are administered to subjects, wherein the subjects can be any member of the animal kingdom. In some embodiments, the subject is a non-human animal. In some embodiments, the non-human subject is an avian (e.g., a chicken or a bird), a reptile, an amphibian, a fish, an insect, and/or a worm. In some embodiments, the non-human subject is a mammal (e.g., a ferret, a rodent, a mouse, a rat, a rabbit, a monkey, a dog, a cat, a sheep, cattle, a primate, and/or a pig).

In some embodiments, the vaccine or immunogenic compositions described herein are administered to a human subject. In particular embodiments, a human subject is 6 months of age or older, 6 months through 35 months of age, at least two years of age, at least 3 years of age, 36 months through 8 years of age, 9 years of age or older, at least 6 months of age and less than 18 years of age, or at least 3 years of age and less than 18 years of age. In some embodiments, the human subject is an infant (less than 36 months). In some embodiments, the human subject is a child or adolescent (less than 18 years of age). In some embodiments, the human subject is elderly (at least 60 years of age or at least 65 years of age). In some embodiments, the human subject is a non-elderly adult (at least 18 years of age and less than 65 years of age). In some embodiments, the methods and uses of the vaccine or immunogenic compositions described herein include prime-boost vaccination strategies. Prime-boost vaccination comprises administering a priming vaccine and then, after a period of time has passed, administering to the subject a boosting vaccine. The immune response is “primed” upon administration of the priming vaccine and is “boosted” upon administration of the boosting vaccine. The priming vaccine can include a vaccine or immunogenic composition comprising the HAs and/or ribonucleic acid molecules as described herein and an optional adjuvant. Likewise, the boosting vaccine can include a vaccine or immunogenic composition comprising the HAs and/or ribonucleic acid molecules as described herein and an optional adjuvant. The priming vaccine or immunogenic composition can be, but need not be, the same as the boosting vaccine. Administration of the boosting vaccine is generally weeks or months after administration of the priming composition, preferably about 2-3 weeks or 4 weeks, or 8 weeks, or 16 weeks, or 20 weeks, or 24 weeks, or 28 weeks, or 32 weeks.

The vaccine or immunogenic composition can be administered using any suitable route of administration, including, for example, parenteral delivery, as discussed above.

Typically, the HAs and/or ribonucleic acid molecules as described herein and optional adjuvant are administered together as components of the same vaccine or immunogenic composition. However, it is not necessary for the HAs and/or ribonucleic acid molecules as described herein to be administered as part of the same vaccine or immunogenic composition. That is, if desired, the HAs and/or ribonucleic acid molecules and optional adjuvant as described herein can be administered to the subject sequentially.

### Representative Embodiments of the Disclosure

1. An immunogenic composition, comprising:
(a) a first influenza virus hemagglutinin (HA) wherein the first influenza virus HA is an H1 HA from a first standard of care influenza virus strain, or a first ribonucleic acid molecule encoding the first influenza virus H1 HA;
(b) a second influenza virus HA wherein the second influenza virus HA is an H3 HA from a second standard of care influenza virus strain, or a second ribonucleic acid molecule encoding the second influenza virus H3 HA;
(c) a third influenza virus HA wherein the third influenza virus HA is from a third standard of care influenza virus strain from the B/Victoria lineage, or a third ribonucleic acid molecule encoding the third influenza virus HA from the B/Victoria lineage;
(d) a fourth influenza virus HA wherein the fourth influenza virus HA is from a fourth standard of care influenza virus strain from the B/Yamagata lineage, or a fourth ribonucleic acid molecule encoding the fourth influenza virus HA from the B/Yamagata lineage; and
(e) one or more machine learning influenza virus HA having a molecular sequence identified or designed from a machine learning model, or one or more ribonucleic acid molecules encoding the one or more machine learning influenza virus HA, wherein the one or more machine learning influenza virus HA are selected from an H1 HA, an H3 HA, an HA from a B/Victoria lineage, an HA from a B/Yamagata lineage, or a combination thereof.

2. The immunogenic composition of embodiment 1, wherein the ribonucleic acid molecule is an mRNA molecule.

3. The immunogenic composition of embodiment 1 or 2, wherein the ribonucleic acid molecule is encapsulated in a lipid-nanoparticle (LNP).

4. The immunogenic composition according to any of embodiments 1-3, wherein the molecular sequence is an amino acid sequence or a nucleic acid sequence.

5. The immunogenic composition according to any of embodiments 1-4, wherein the one or more machine learning influenza virus HA comprise a wild type influenza virus HA molecular sequence.

6. The immunogenic composition according to any of embodiments 1-5, wherein the one or more machine learning influenza virus HA comprise a non-wild type influenza virus HA molecular sequence.

7. The immunogenic composition according to any of embodiments 1-6, wherein the one or more machine learning influenza virus HA is a recombinant influenza virus HA.

8. The immunogenic composition according to any of embodiments 1-6, wherein the one or more machine learning influenza virus HA is present in an inactivated influenza virus, optionally a split-inactivated virus.

9. The immunogenic composition according to any of embodiments 1-6, comprising a ribonucleic acid molecule encoding at least one of the one or more machine learning influenza virus HA.

10. The immunogenic composition according to any of embodiments 1-9, wherein the one or more machine learning influenza virus HAs is a fifth influenza virus HA, wherein the fifth influenza virus HA is an H3 HA, and wherein the fifth influenza H3 HA is antigenically dissimilar than the second influenza H3 HA.

11. The immunogenic composition according to any of embodiments 1-9, wherein the one or more machine learning influenza virus HAs is a fifth influenza virus HA, wherein the fifth influenza virus HA is an H3 HA, and wherein the fifth influenza H3 HA enhances a protective immune response induced by the second influenza H3 HA.

12. The immunogenic composition according to any of embodiments 1-9, wherein the one or more machine learning influenza virus HAs is a fifth influenza virus HA, wherein the fifth influenza virus HA is an H3 HA, and wherein the fifth influenza H3 HA broadens a protective immune response induced by the second influenza H3 HA.

13. The immunogenic composition according to any of embodiments 1-9, wherein the one or more machine learning influenza virus HAs is a fifth influenza virus HA, wherein the fifth influenza virus HA is an H3 HA, and wherein the fifth influenza H3 HA is from a different clade than the second influenza H3 HA.

14. The immunogenic composition according to any of embodiments 1-9, wherein the one or more machine learning influenza virus HAs is a fifth influenza virus HA, wherein the fifth influenza virus HA is an H3 HA, and wherein the fifth influenza H3 HA is antigenically similar to the second influenza H3 HA.

15. The immunogenic composition according to any of embodiments 1-9, wherein the one or more machine learning influenza virus HAs is a fifth influenza virus HA, wherein the fifth influenza virus HA is an H3 HA, and wherein the fifth influenza H3 HA is from a same clade as the second influenza H3 HA.

16. The immunogenic composition according to any of embodiments 1-9, wherein the one or more machine learning influenza virus HA is a fifth influenza virus HA, wherein the fifth influenza virus HA is an H1 HA, and wherein the fifth influenza H1 HA is antigenically dissimilar than the first influenza H1 HA.

17. The immunogenic composition according to any of embodiments 1-9, wherein the one or more machine learning influenza virus HA is a fifth influenza virus HA, wherein the fifth influenza virus HA is an H1 HA, and wherein the fifth influenza H1 HA enhances a protective immune response induced by the first influenza H1 HA.

18. The immunogenic composition according to any of embodiments 1-9, wherein the one or more machine learning influenza virus HA is a fifth influenza virus HA, wherein the fifth influenza virus HA is an H1 HA, and wherein the fifth influenza H1 HA broadens a protective immune response induced by the first influenza H1 HA.

19. The immunogenic composition according to any of embodiments 1-9, wherein the one or more machine learning influenza virus HA is a fifth influenza virus HA, wherein the fifth influenza virus HA is an H1 HA, and wherein the fifth influenza H1 HA is from a different clade than the first influenza H1 HA.

20. The immunogenic composition according to any of embodiments 1-9, wherein the one or more machine learning influenza virus HAs is a fifth influenza virus HA, wherein the fifth influenza virus HA is an H1 HA, and wherein the fifth influenza H1 HA is antigenically similar to the first influenza H1 HA.

21. The immunogenic composition according to any of embodiments 1-9, wherein the one or more machine learning influenza virus HAs is a fifth influenza virus HA, wherein the fifth influenza virus HA is an H1 HA, and wherein the fifth influenza H1 HA is from a same clade as the first influenza H1 HA.

22. The immunogenic composition according to any of embodiments 1-9, wherein the one or more machine learning influenza virus HAs is a fifth influenza virus HA, wherein the fifth influenza virus HA is an H3 HA from the 3C.2A clade.

23. The immunogenic composition according to any of embodiments 1-9, wherein the one or more machine learning influenza virus HAs is a fifth influenza virus HA, wherein the fifth influenza virus HA is an H3 HA from the 3C.3A clade.

24. The immunogenic composition according to any of embodiments 1-15, further comprising a sixth influenza virus HA.

25. The immunogenic composition according to embodiment 24, wherein the sixth influenza virus HA is an H1 HA having a molecular sequence identified or designed from a machine learning model, or a ribonucleic acid molecule encoding the sixth influenza virus HA.

26. The immunogenic composition according to embodiment 25, wherein the sixth influenza H1 HA is antigenically dissimilar than the first influenza H1 HA, wherein the sixth influenza H1 HA enhances a protective immune response induced by the first influenza H1 HA, wherein the sixth influenza H1 HA broadens a protective immune response induced by the first influenza H1 HA, wherein the sixth influenza H1 HA is from a different clade than the first influenza H1 HA, wherein the sixth influenza H1 HA is from a same clade as the first influenza H1 HA, or wherein the sixth influenza H1 HA is antigenically similar to the first influenza H1 HA.

27. The immunogenic composition according to any one of embodiments 24-26, further comprising a seventh influenza virus HA from the B/Victoria lineage having a molecular sequence identified or designed from a machine learning model, or a ribonucleic acid molecule encoding the seventh influenza virus HA.

28. The immunogenic composition according to any of embodiments 24-27, further comprising an eighth influenza virus HA from the B/Yamagata lineage having a molecular sequence identified or designed from a machine learning model, or a ribonucleic acid molecule encoding the eighth influenza virus HA.

29. The immunogenic composition according to any of embodiments 1-28, wherein the machine learning model is trained to predict a biological response.

30. The immunogenic composition according to embodiment 29, wherein the biological response is a human, ferret, or mouse biological response.

31. The immunogenic composition according to embodiment 29 or 30, wherein the biological response comprises a hemagglutinin inhibition assay (HAI), antibody forensics (AF), or neutralization assay.

32. The immunogenic composition according to any one of embodiments 1-31, wherein each of the first, second, third, and fourth influenza virus HA is a recombinant influenza virus HA.

33. The immunogenic composition according to any one of embodiments 1-31, wherein each of the first, second, third, and fourth influenza virus HA is present in an inactivated influenza virus.

34. The immunogenic composition according to any one of embodiments 1-31, comprising the first, second, third, and fourth influenza virus HA as ribonucleic acid molecules.

35. The immunogenic composition according to any one of embodiments 7-34, wherein each of the recombinant influenza virus HA is produced by a baculovirus expression system in cultured insect cells.

36. The immunogenic composition according to any of embodiments 1-35, wherein the first influenza virus HA is an H1 HA from an H1N1 influenza virus strain and the second influenza virus HA is an H3 HA from an H3N2 influenza virus strain.

37. The immunogenic composition according to any of embodiments 1-36, wherein the composition further comprises an adjuvant.

38. The immunogenic composition according to embodiment 37, wherein the adjuvant comprises a squalene-in-water adjuvant or a liposome-based adjuvant.

39. The immunogenic composition according to embodiment 38, wherein the squalene-in-water adjuvant comprises AF03.

40. The immunogenic composition according to embodiment 38, wherein the liposome-based adjuvant comprises SPA14.

41. The immunogenic composition according to any of embodiments 1-40, wherein each ribonucleic acid molecule comprises one or more modified nucleotides.

42. The immunogenic composition according to any of embodiments 1-41, wherein the composition is formulated for intramuscular injection.

43. The immunogenic composition according to any of embodiments 1-42, wherein the ribonucleic acid molecule is encapsulated in an LNP comprising a cationic lipid, a PEGylated lipid, a cholesterol-based lipid, and a helper lipid.

44. A method of immunizing a subject against influenza virus, the method comprising administering to the subject an immunologically effective amount of the immunogenic composition of any one of embodiments 1-43.

45. The method of embodiment 44, wherein the method prevents influenza virus infection in the subject.

46. The method of embodiment 44 or 45, wherein the method raises a protective immune response in the subject.

47. The method of embodiment 46, wherein the protective immune response comprises an HA antibody response.

48. The method of any one of embodiments 44-47, wherein the subject is human.

49. The method of any one of embodiments 44-48, wherein the immunogenic composition is administered intramuscularly, intradermally, subcutaneously, intravenously, intranasally, by inhalation, or intraperitoneally.

50. The method of any one of embodiments 44-49, wherein the method treats or prevents disease caused by either or both a seasonal and a pandemic influenza strain.

51. The method of any one of embodiments 44-50, wherein the subject is human and the human is 6 months of age or older, 6 to 35 months of age, at least 2 years of age, at least 3 years of age, less than 18 years of age, at least 18 years of age, at least 60 years of age, at least 65 years of age, at least 6 months of age and less than 18 years of age, at least 3 years of age and less than 18 years of age, or at least 18 years of age and less than 65 years of age.

52. A method of reducing one or more symptoms of influenza virus infection, the method comprising administering to a subject a prophylactically effective amount of the immunogenic composition of any one of embodiments 1-43.

53. The method of any one of embodiments 44-52 comprising administering to the subject two doses of the immunogenic composition with an interval of 2-6 weeks, optionally 4 weeks.

54. A vaccine composition comprising the immunogenic composition according to any one of embodiments 1-43.

55. The method of any one of embodiments 44-53, wherein the immunogenic composition is a vaccine composition.

The present disclosure will be more fully understood by reference to the following Examples.

### EXAMPLES

The following example is to be considered illustrative and not limiting on the scope of the disclosure described above.

Animal experiments were carried out in compliance with the Public Health Service (PHS) Policy on Humane Care and Use of Laboratory Animals and the Guide for the Care and Use of Laboratory Animals and were conducted with approved animal protocols from the Sanofi Institutional Animal Care and Use Committee (IACUC). All animals were housed under specified pathogen-free conditions with food and water ad libitum.

Hemagglutinin-Inhibition (HAI) Assay: Sera were treated with receptor-destroying enzyme (RDE; Denka Seiken, Co., Japan) to inactivate nonspecific inhibitors prior to HAI assay. RDE-treated sera were serially diluted (2-fold dilutions) in v-bottom microtiter plates. An equal volume of each virus from the HAI readout panel was added to each well (4 hemagglutinating units (HAU) per well). The homologous virus panels used are described in the Examples below and unless otherwise indicated were grown in eggs. The plates were covered and incubated at room temperature for 20 minutes (or 45-60 min), followed by the addition of 1% mixture of chicken erythrocytes (red blood cells; CRBC) or 0.5% mixture of turkey red blood cells (TRBC) (Lampire Biologicals) in PBS. The plates were mixed by agitation and covered, and the RBCs were allowed to settle for approximately 30 minutes to 1 hour at room temperature. The HAI titer was determined by the reciprocal dilution of the last well which contained non-agglutinated RBCs.

HINT mNT Influenza Protocol: Neutralization titers against influenza strains were measured as adapted from Jorquera, P. A. et al, Insights into the antigenic advancement of influenza A (H3N2) viruses, 2011-2018, Sci. Reports 9, 2676 (2019). Briefly, serial 2-fold dilutions of RDE treated sera from 1:20 to 1:2,560 were mixed with an equal volume of virus, about 1000 focus forming units (FFU), and incubated for 60 minutes at 37° C. After incubation, an MDCK-SIAT1 cell suspension was added to the virus:sera mixture and incubated for about 22 hrs. The monolayers were fixed with methanol and prepared for staining. Wells were then incubated with anti-influenza monoclonal antibody against nucleoprotein (NP), followed by an ALEXA FLUOR® 488 -conjugated secondary antibody Thermo Fisher Scientific; Waltham, MA). Cells were washed and plates scanned on CTL IMMUNOSPOT® Cell Imaging v2 (CTL, Cleveland, Ohio). Counts from the plate were transferred into Graphpad Prism software and neutralization titer 50 (NT50) was calculated using a sigmoidal dose-response, variable slope, non-linear progression. The NT50 titer of a sera sample which inhibited virus infection by 50% of the virus input only control wells were a calculated titer from the sigmoidal curve. The assay does not include trypsin and measures inhibition of virus entry as compared to virus input control wells with no sera. The counts were individual infected cells, and the assay is suitable for all live virus subtypes, including H1, H3, B/Victoria, and B/Yamagata.

### Example 1—Immune Response to Multiple H3 HA Strain Administration

To determine the effect on an HA immune response, multiple H3 HAs were administered in the naïve ferret model. Ferrets were infected either with a single H3N2 inactivated virus or a cocktail of two H3N2 inactivated viruses to determine if the antibody responses elicited by the cocktail showed increase breadth over the responses elicited by the single virus against an antigenically diverse virus read out panel.

The viruses selected for co-infection were chosen either from the same clade 3C.2A (similar viruses) or divergent clades 3C.2A and 3C.3A (dissimilar viruses) and are shown below in table 1.

TABLE 1
H3N2 Viruses for Infection

| Strain | Clade Designation | Category |
|---|---|---|
| A/HONGKONG/45/2019 | 3C.2A1B/135K/137F | Single virus |
| A/ALASKA/43/2019 | 3C.2A1B/131K/197R | Single virus |
| A/KANSAS/14/2017 | 3C.3A | Single virus |
| A/HONGKONG/45/2019 + | 3C.2A1B/135K/137F + | Virus cocktail |
| A/ALASKA/43/2019 | 3C.2A1B/131K/197R | (antigenically similar) |
| A/HONGKONG/45/2019 + | 3C.2A1B/135K/137F + 3C.3A | Virus cocktail |
| A/KANSAS/14/2017 | | (antigenically |
| | | dissimilar) |

The read-out panel contained viruses from the 2016 to 2019 time period and are representative of circulating strains from two antigenically distinct clades, 3C.2A and 3C.3A, to assess cross clade coverage. The following seven 3C.2A viruses were used in the read-out panel: A/Valladolid/182/2017, A/Alaska/43/2019, A/Bangladesh/3190613015/2019, A/Hongkong/45/2019, A/Victoria/617/2017, A/Peru/9519/2019, and A/Singapore/INFIMH-16-0019/2016. The following five 3C.3A viruses were used in the read-out panel: A/Kansas/14/2017, A/Brisbane/34/2018, A/Mexico/2356/2019, A/Suriname/0902/2019, and A/Indiana/08/2018.

Naïve ferrets (2 per group) were inoculated intranasally with (1) A/Hongkong/45/2019 alone; (2) A/Alaska/43/2019 alone; (3) A/Kansas/14/2017 alone; (4) a 1:1 combination of A/Hongkong/45/2019 and A/Alaska/43/2019; or (5) a 1:1 combination of A/Hongkong/45/2019 and A/Kansas/14/2017. Each virus was given at the same dose, 5 log₁₀ focus forming units (FFU). Blood was collected on Day −8, the ferrets were immunized on Day 0, and blood was collected again on Day 14.

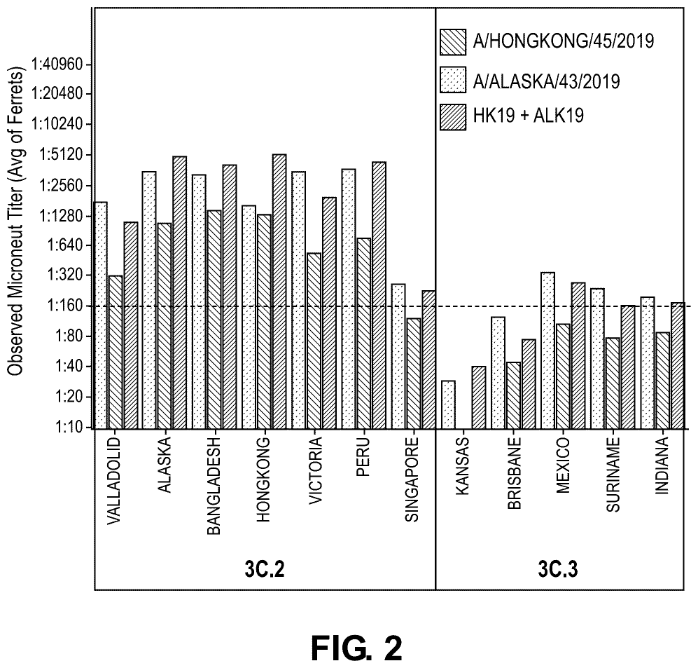

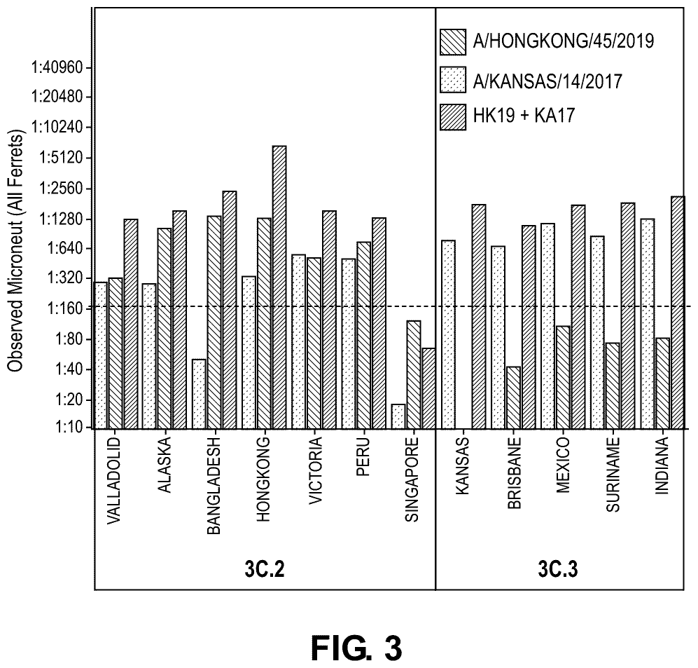

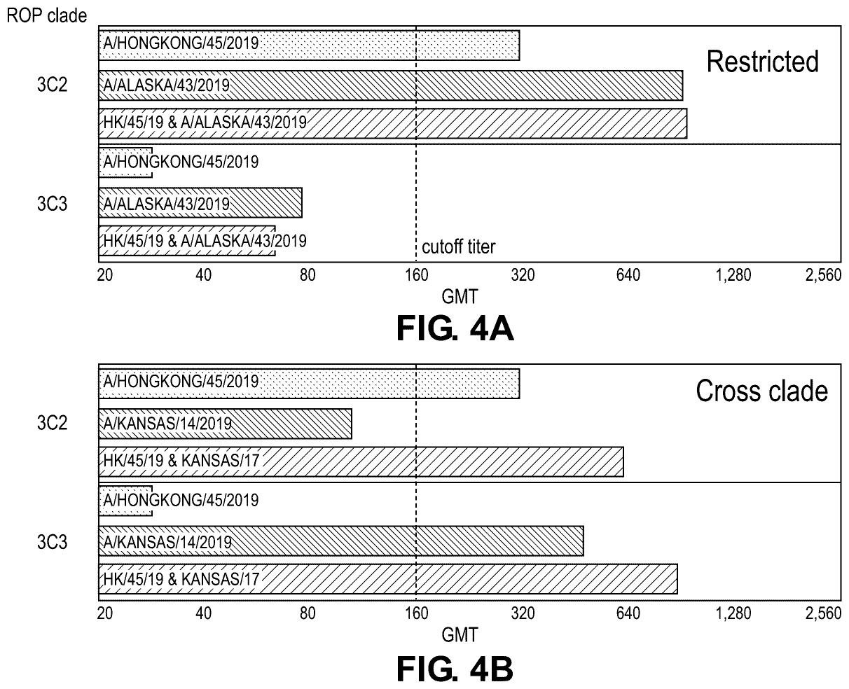

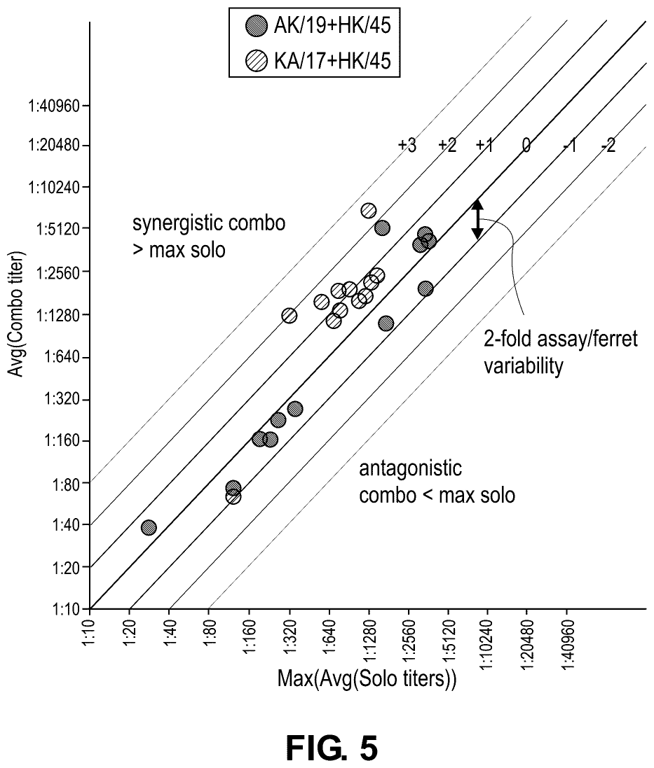

High microneutralization titers were observed from ferrets infected with the combination of A/HONGKONG/45/2019+A/KANSAS/14/2017 viruses against read out viruses covering the 3C.2A and 3C.3A clades. See FIG. 3 and FIG. 4B. In contrast, ferrets infected with an antigenically similar virus cocktail containing a combination of A/HONGKONG/45/2019+A/ALASKA/43/2019 exhibited more clade restricted responses with high titers observed to read out strains from the 3C.2A clade. See FIG. 2 and FIG. 4B. The combination of virus cocktails does not appear to interfere with the responses elicited to each individual strain within the cocktail and are like that observed as with the single infections. See FIG. 5.

The mixture of H3 HAs from opposite clades (3C.2A and 3C.3A) showed an additive effect of the antibody response that exhibited cross clade breadth with the highest magnitude of neutralization titers. Thus, the overall response observed in this 3C.2A+3C.3A cocktail were like that observed for each single infection of 3C.2A and 3C.3A virus. The virus mixtures from the same 3C.2A clade did not elicit the same magnitude of titers expanding into the 3C.3A clade as the 3C.2A+3C.3A virus cocktail, however this mixture did show that the addition of the A/ALASKA/43/2019 virus to the standard of care virus, A/HONGKONG/45/2019, did improve the antibody titers across the entire read out panel. See FIG. 2, comparing (1) A/Hongkong/45/2019 titers to (2) titers from the combination of A/Hongkong/45/2019 and A/Alaska/43/2019. The data shows combining different H3 HAs can increase breadth, allowing for improved coverage of antigenically diverse influenza viruses.

### Example 2—Immunogenicity in the Naïve Ferret Model Evaluating Quadrivalent and Pentavalent Influenza Vaccines

The objective of the study was to determine if mixing two H3 HAs delivered in a modified, non-replicating (MNR) mRNA formulation elicited an additive, synergistic, or antagonist effect on the HA immune response in the naïve ferret model. The study further evaluated the feasibility of a pentavalent influenza vaccine (PIV) comprising an additional H3 strain to broaden coverage as compared to a quadrivalent influenza vaccine (QIV) without the additional strain.

Naïve ferrets used to assess multivalent vaccine immunogenicity were vaccinated twice 21 days apart (on Day 0 and on Day 21) with one of the following 10 groups, as described in Table 2 below:

Group (1) a mixture of five mRNAs encoding HA antigens, four of which were selected from the 2021-2022 northern hemisphere WHO standard of care (WHO SOC) strains (H1, H3, BVic, and BYam) (specifically from strains A/Wisconsin/588/2019 (H1N1), A/Tasmania/503/2020 (H3N2), B/Washington/02/2019 ,(Victoria lineage) and B/Phuket/3073/2013 (Yamagata lineage)), and one of which was selected via machine learning to provide clade H3C.2A protection (specifically, wild-type A/Norway/2629/2015), with the HA mRNA from each strain present in an amount of 15 μg;

Group (2) a mixture of five mRNAs encoding HA antigens, four of which were the WHO SOC strains and one of which was selected via machine learning to provide clade H3C.2A protection (specifically, non-wild type A/Design/H3S25/2019), with the HA mRNA from each strain present in an amount of 15 μg;

Group (3) a mixture of five mRNAs encoding HA antigens, four of which were the WHO SOC strains and one of which was selected via machine learning to provide clade H3C.3A protection (specifically, wild type A/Washington/526/2019), with the HA mRNA from each strain present in an amount of 15 μg;

Group (4) a mixture of five mRNAs encoding HA antigens, four of which were the WHO SOC strains noted above and one of which was the additional WHO SOC strain A/Kansas/14/2017, selected to provide clade H3C.3A protection, with the HA mRNA from each strain present in an amount of 15 μg;

Group (5) a mixture of five mRNAs encoding HA antigens, four of which were the WHO SOC strains noted above and one of which was an additional 2019-2020 northern hemisphere WHO SOC strain A/Kansas/14/2017, with HA mRNAs from each of H1, BYam, and BVic present in an amount of 15 μg each and mRNAs of the two H3 strains (A/Tasmania/503/2020 and A/Kansas/14/2017) each present in an amount of 7.5 μg;

Group (6) a mixture of four mRNAs encoding the WHO SOC strains, with mRNA from each of the H1, BYam, and BVic strains present in an amount of 15 μg and mRNA from the H3 strain present in an amount of 30 μg;

Group (7) a mixture of four mRNAs encoding the WHO SOC strains, with mRNA from each of the four strains present in an amount of 15 μg;

Group (8) a mixture of four recombinant HA proteins selected from the WHO SOC strains, with recombinant HA from each of the four strains present in an amount of 45 μg;

Group (9) a mixture of four inactivated viruses selected from the WHO SOC strains, with each of the four strains present in an amount of 60 μg; and

Group (10) phosphate buffered saline (PBS).

All vaccine formulations with the exception of Groups 8-10 contained mono-encapsulated (single subtype/LNP) MNR mRNA HAs that were combined into a single formulation prior to immunization to produce the desired vaccine combination.

Ferrets were immunized intramuscularly on Day 0 and Day 21, and humoral responses were evaluated one month after the second immunization on Day 49, using a microneutralization (mNT) assay to measure functional HA antibody response.

TABLE 2
Naïve Ferret Study Groups

| | | | μg per | μg per |
|---|---|---|---|---|
| | | Machine | strain | strain HA |
| | | Learning | HA | protein |
| | | Immunogen | mRNA (μg | (μg HA |
| Group | Vaccine type | H3 | mRNA | protein |
| # | (Clade) | strain | Total) | total) |
| 1 | Quadrivalent WHO | A/Norway/ | 15 (75) | |
| | SOC mRNA (3C.2A) + | 2629/2015 | | |
| | Machine Learning | | | |
| | WT (3C.2A) | | | |
| 2 | Quadrivalent WHO | A/Design/ | 15 (75) | |
| | SOC mRNA (3C.2A) + | H3S25/2019 | | |
| | Machine Learning | | | |
| | non-WT (3C.2A) | | | |
| 3 | Quadrivalent WHO | A/Washington/ | 15 (75) | |
| | SOC mRNA (3C.2A) + | 526/2019 | | |
| | Machine Learning | | | |
| | WT (3C.3A) | | | |
| 4 | Quadrivalent WHO | — | 15 (75) | |
| | SOC mRNA (3C.2A) + | | | |
| | Additional WHO | | | |
| | SOC mRNA | | | |
| | A/Kansas (3C.3A) | | | |
| 5 | Quadrivalent WHO | — | 7.5 H3 (15 H3 | |
| | SOC mRNA (3C.2A) + | | total); | |
| | Additional WHO | | 15 H1; 15 | |
| | SOC mRNA | | B/Yam; 15 | |
| | A/Kansas (3C.3A) | | B/Vic (60) | |
| 6 | Quadrivalent WHO | — | 30 H3; | |
| | SOC mRNA (3C.2A) | | 15 H1; 15 | |
| | | | B/Yam; 15 | |
| | | | B/Vic (75) | |
| 7 | Quadrivalent WHO | — | 15 (60) | |
| | SOC mRNA (3C.2A) | | | |
| 8 | Quadrivalent WHO | — | — | 45 (180) |
| | SOC recombinant | | | |
| | proteins (3C.2A) | | | |
| 9 | High-dose | — | — | 60 (240) |
| | quadrivalent | | | |
| | inactivated virus | | | |
| | (3C.2A) | — | — | — |
| 10 | PBS | | | |

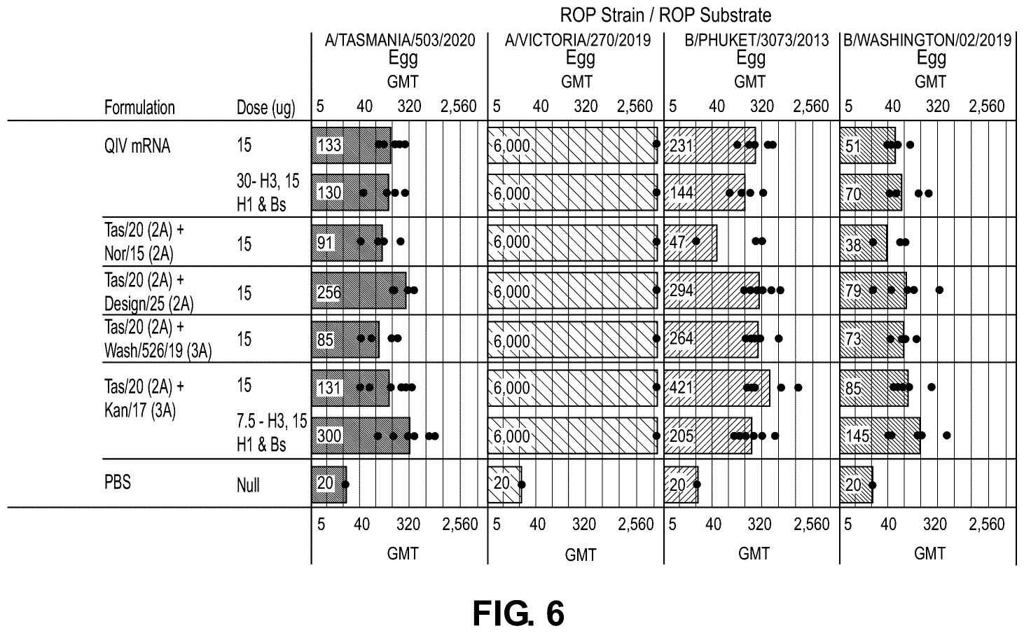

For each group, n=6 ferrets. mNT antibody titers were measured against the following egg amplified influenza virus strains: A/Tasmania/503/2020, A/Victoria/2570/2019, B/Phuket,/3073/2013 and B/Washington/02/2019, and the geometric mean titer (GMT) values were calculated. The results are reported in FIG. 6.

The titer cap for H1 (A/Victoria/2570/2019) was between 6,000-7,000. As shown above and in FIG. 6, ferrets receiving quadrivalent mRNA vaccine (Groups 6 and 7) produced functional antibody response by Day 49 directed against all 4 homologous influenza subtypes H1N1, H3N2, B/Yamagata, and B/Victoria. The H1 responses were robust for all of Groups 1-9, wherein titers were capped at 1:6,000.

For the B/Victoria responses in ferrets receiving PIV containing 15 μg of HA mRNA per strain (Groups 1-4), the neutralization titers elicited against B/Washington/02/2019 were comparable to those elicited from ferrets receiving the QIV containing 15 μg of HA mRNA per strain (Group 7). Likewise, similar B/Yamagata responses were observed for Groups 2-4 and 7, with the exception being that, for Group 1, four of the ferrets did not generate homologous neutralization titers against B/Phuket/3073/2013, and overall titers were significantly lower than those for Group 7 (p<0.0001 for mixed model analysis). Without being bound by theory, it is possible that the results indicate a technical issue with immunization or a strain-specific effect, as the addition of the H3 strain in the other PIV groups did not result in any significant drop in mNT titers.

There were no statistically significant differences in antibody response measured at Day 49 between the 15 μg dose of H3 A/Tasmania/503/2020 or the 30 μg dose of H3 A/Tasmania/503/2020 QIV mRNA vaccine Groups 6 and 7 (p=0.95, mixed model analysis). This suggests that doubling the H3 antigen dose did not increase the magnitude of the neutralizing antibody titers in this dose range.

Ferrets immunized with a 7.5 μg dose of H3 in the PIV formulation (Group 5) showed significantly higher A/Tasmania/503/2020 mNT titers than the ferrets immunized with the QIV formulation (Groups 6 and 7, where the H3 component was dosed at 15 μg and 30 μg, respectively) (p <0.05; mixed model analysis).

Groups 1-4, each of which contained two H3 strains in a PIV formulation (30 μg total H3), showed comparable homologous A/Tasmania/503/2020 mNT titers that were within 2-fold of the QIV control GMT titers (Groups 6 and 7) (no significant differences by mixed model analysis). The ferrets in Group 5 immunized with the lower dose (7.5 μg of each H3 strain) elicited a significantly higher homologous response than that for Groups 6 and 7, wherein the response was 2.3 fold higher (p<0.05 by mixed model analysis). Significantly, the data indicate the addition of an H3 strain to create a PIV formulation did not hinder the homologous mNT responses elicited by the H1, H3, B/Victoria, or B/Yamagata WHO SOC strains.

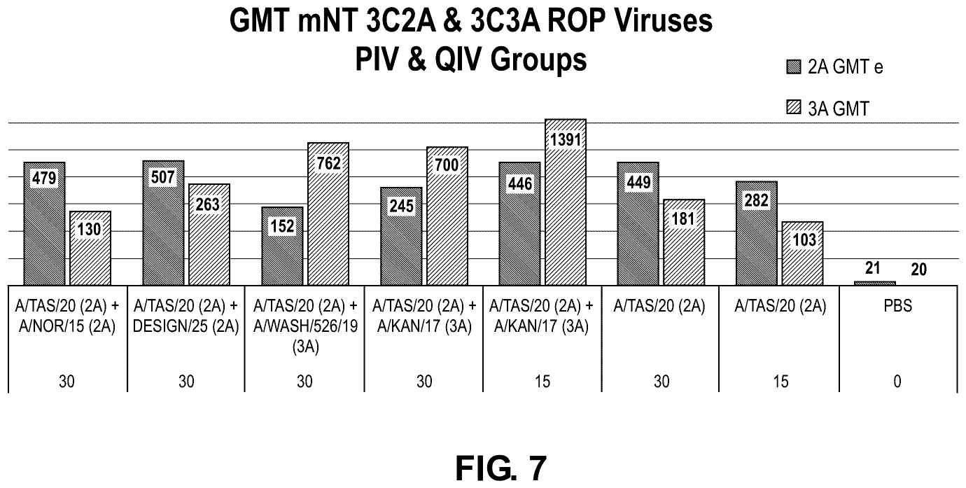

The study also addressed if the coverage of the H3 antigenic space could be broadened by the addition to a QIV of a second H3 strain, either from a machine learning selection or from a previous WHO SOC selection, to create a PIV, e.g., Groups 1-5. While the WHO 2021-2022 SOC H3 strain A/Tasmania/503/2020 (a 3C.2A circulating clade) was part of the QIV formulation, two alternative H3 clade 3C.2A strains were included in PIV formulations as the fifth strain: the machine learning selected, wild type strain A/Norway/2629/2015 (Group 1) and the machine learning selected, non-wild type strain A/Design/H3S25/2019 (Group 2). These PIV formulations containing two 3C.2A H3 strains showed slightly increased GMT mNT titers as compared to the single 3C.2A formulation in the QIV (Group 7) and demonstrated no negative interference on heterologous responses when similar HAs were co-administered in naïve ferrets. The results are shown below in Table 3. See also FIG. 7, showing mNT GMT values across 3C.2A and 3C.3A strains for both PIV (Groups 1-5) and QIV (Groups 6 and 7) vaccine formulations.

TABLE 3
Neutralizing GMT Titers across 3C.2A and 3C.3A PIV
and QIV vaccine formulations

| Group | 3C.2A GMT | 3C.3A GMT |
|---|---|---|
| 1 | 479 | 130 |
| 2 | 507 | 263 |
| 3 | 152 | 762 |
| 4 | 245 | 700 |
| 5 | 446 | 1391 |
| 6 | 449 | 181 |
| 7 | 282 | 103 |
| 10 | 21 | 20 |

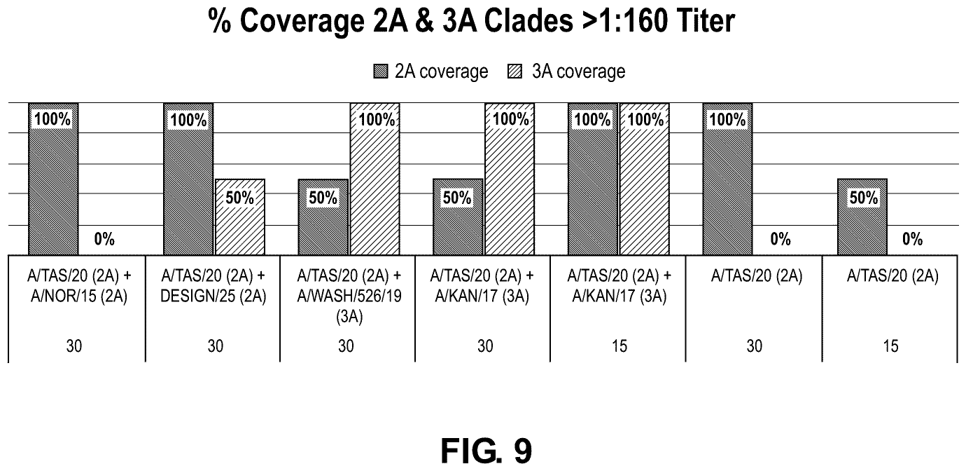

In general, ferrets immunized with two H3s from the 3C.2A clade (Groups 1 and 2) did not expand breadth into the 3C.3A space; however, the machine learning designed H3 strain A/Design/H3S25/2019 did efficiently neutralize 50% of the 3C.3A viruses, unlike the QIV control Groups 6 and 7. See FIG. 9.

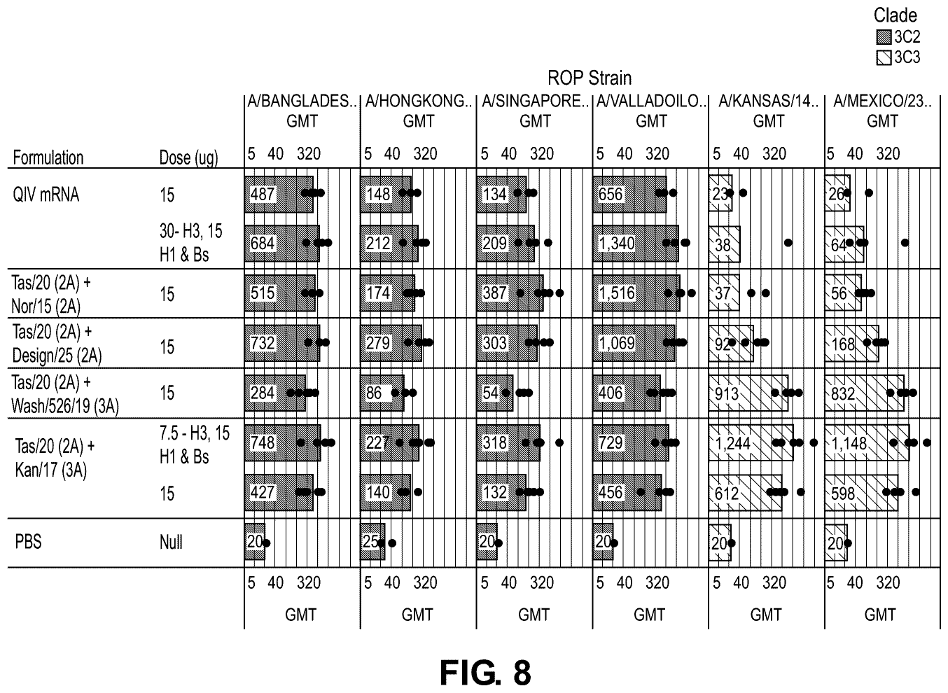

Alternatively, addition of an H3 strain from the 3C.3A clade to the quadrivalent vaccine's H3 3C.2A strain, as was done in Groups 3-5, showed at least an additive effect of the antibody response when compared to the heterologous multi-clade 3C.2A and 3C.3A viruses, as shown in FIG. 8 and reported below in Table 4.

For each of Groups #1-7, mNT titers were measured for the following cell amplified influenza virus strains: A/Bangladesh/3190613015/2019; A/Mexico/2356/2019; A/Valladolid/182/2017; A/Brisbane/75/2019; A/Tasmania/503/2020; A/HongKong/45/2019; A/Kansas/14/2017; and A/Singapore/Infimh160019/2016. Both A/Mexico/2356/2019 and A/Kansas/14/2017 are clade 3C.3A. A/Bangladesh/3190613015/2019; A/Brisbane/75/2019; A/Tasmania/503/2020; and A/HongKong/45/2019 are clade/subclade 3C.2A1b. A/Valladolid/182/2017 is clade/subclade 3C.2A4, and A/Singapore/Infimh160019/2016 is clade/subclade 3C.2A1. The results are reported below in Table 4.

TABLE 4
Geometric Mean Titers (GMT) for Multivalent Vaccinations (Day 49)

| | GMT for HINT mNT Assay | | | | | |
|---|---|---|---|---|---|---|
| Group | Clade 3C.2A | | | | Clade 3C.3A | |
| # | A/Bangladesh | A/HongKong | A/Singa | A/Valladolid | A/Kansas | A/Mexico |
| 1 | 515 | 174 | 387 | 1,516 | 37 | 56 |
| 2 | 732 | 279 | 303 | 1,069 | 92 | 168 |
| 3 | 284 | 86 | 54 | 406 | 913 | 832 |
| 4 | 427 | 140 | 132 | 456 | 612 | 598 |
| 5 | 748 | 227 | 318 | 729 | 1,244 | 1,148 |
| 6 | 684 | 212 | 209 | 1,340 | 38 | 64 |
| 7 | 487 | 148 | 134 | 656 | 23 | 26 |
| 10 | 20 | 25 | 20 | 20 | 20 | 20 |

As shown above in Table 4 and FIG. 8, in addition to increasing the magnitude of mNT titers, the coverage of strains in the divergent 3C.3A clade was 100% in the PIV formulation Groups 3-5, whereas no coverage of the 3C.3A clade was observed in the QIV formulation Groups 6 and 7, as shown in FIG. 9. Maximizing coverage in a multiclade season with the delivery of two different H3 HAs demonstrates the potential of improving the efficacy of standard of care quadrivalent vaccine formulations.

It is also noted that, as used in this disclosure and the appended claims, the singular forms “a”, “an”, and “the” include plural referents unless the context clearly dictates otherwise. Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination). Ranges may be expressed herein as from about one particular value, and/or to about another particular value. When such a range is expressed, another aspect includes from the one particular value and/or to the other particular value. Similarly, when values are expressed as approximations, by use of the antecedent about, it will be understood that the particular value forms another aspect. It will be further understood that the endpoints of each of the ranges are significant both in relation to the other endpoint, and independently of the other endpoint. All references cited in this disclosure are hereby incorporated by reference in their entirety.

What is claimed is:

1. An immunogenic composition, comprising:
(a) a first influenza virus hemagglutinin (HA) wherein the first influenza virus HA is an H1 HA from a first standard of care influenza virus strain, or a first ribonucleic acid molecule encoding the first influenza virus H1 HA;
(b) a second influenza virus HA wherein the second influenza virus HA is an H3 HA from a second standard of care influenza virus strain, or a second ribonucleic acid molecule encoding the second influenza virus H3 HA;
(c) a third influenza virus HA wherein the third influenza virus HA is from a third standard of care influenza virus strain from the B/Victoria lineage, or a third ribonucleic acid molecule encoding the third influenza virus HA from the B/Victoria lineage;
(d) a fourth influenza virus HA wherein the fourth influenza virus HA is from a fourth standard of care influenza virus strain from the B/Yamagata lineage, or a fourth ribonucleic acid molecule encoding the fourth influenza virus HA from the B/Yamagata lineage; and
(e) one or more machine learning influenza virus HA having a molecular sequence identified or designed from a machine learning model, or one or more ribonucleic acid molecules encoding the one or more machine learning influenza virus HA, wherein the one or more machine learning influenza virus HA are selected from an H1 HA, an H3 HA, an HA from a B/Victoria lineage, an HA from a B/Yamagata lineage, or a combination thereof.

2. The immunogenic composition according to claim 1, wherein the ribonucleic acid molecule is an mRNA molecule.

3. The immunogenic composition according to claim 1 or 2, wherein the ribonucleic acid molecule is encapsulated in a lipid-nanoparticle (LNP).

4. The immunogenic composition according to any of the preceding claims, wherein the molecular sequence is an amino acid sequence or a nucleic acid sequence.

5. The immunogenic composition according to any of the preceding claims, wherein the one or more machine learning influenza virus HA comprise a wild type influenza virus HA molecular sequence.

6. The immunogenic composition according to any of the preceding claims, wherein the one or more machine learning influenza virus HA comprise a non-wild type influenza virus HA molecular sequence.

7. The immunogenic composition according to any of the preceding claims, wherein the one or more machine learning influenza virus HA is a recombinant influenza virus HA.

8. The immunogenic composition according to any of claims 1-6, wherein the one or more machine learning influenza virus HA is present in an inactivated influenza virus, optionally a split-inactivated virus.

9. The immunogenic composition according to any of claims 1-6, comprising a ribonucleic acid molecule encoding at least one of the one or more machine learning influenza virus HA.

10. The immunogenic composition according to any of claims 1-9, wherein the one or more machine learning influenza virus HAs is a fifth influenza virus HA, wherein the fifth influenza virus HA is an H3 HA, and wherein the fifth influenza H3 HA is antigenically dissimilar than the second influenza H3 HA.

11. The immunogenic composition according to any of claims 1-9, wherein the one or more machine learning influenza virus HAs is a fifth influenza virus HA, wherein the fifth influenza virus HA is an H3 HA, and wherein the fifth influenza H3 HA enhances a protective immune response induced by the second influenza H3 HA.

12. The immunogenic composition according to any of claims 1-9, wherein the one or more machine learning influenza virus HAs is a fifth influenza virus HA, wherein the fifth influenza virus HA is an H3 HA, and wherein the fifth influenza H3 HA broadens a protective immune response induced by the second influenza H3 HA.

13. The immunogenic composition according to any of claims 1-9, wherein the one or more machine learning influenza virus HAs is a fifth influenza virus HA, wherein the fifth influenza virus HA is an H3 HA, and wherein the fifth influenza H3 HA is from a different clade than the second influenza H3 HA.

14. The immunogenic composition according to any of claims 1-9, wherein the one or more machine learning influenza virus HAs is a fifth influenza virus HA, wherein the fifth influenza virus HA is an H3 HA, and wherein the fifth influenza H3 HA is antigenically similar to the second influenza H3 HA.

15. The immunogenic composition according to any of claims 1-9, wherein the one or more machine learning influenza virus HAs is a fifth influenza virus HA, wherein the fifth influenza virus HA is an H3 HA, and wherein the fifth influenza H3 HA is from a same clade as the second influenza H3 HA.

16. The immunogenic composition according to any of claims 1-9, wherein the one or more machine learning influenza virus HA is a fifth influenza virus HA, wherein the fifth influenza virus HA is an H1 HA, and wherein the fifth influenza H1 HA is antigenically dissimilar than the first influenza H1 HA.

17. The immunogenic composition according to any of claims 1-9, wherein the one or more machine learning influenza virus HA is a fifth influenza virus HA, wherein the fifth influenza virus HA is an H1 HA, and wherein the fifth influenza H1 HA enhances a protective immune response induced by the first influenza H1 HA.

18. The immunogenic composition according to any of claims 1-9, wherein the one or more machine learning influenza virus HA is a fifth influenza virus HA, wherein the fifth influenza virus HA is an H1 HA, and wherein the fifth influenza H1 HA broadens a protective immune response induced by the first influenza H1 HA.

19. The immunogenic composition according to any of claims 1-9, wherein the one or more machine learning influenza virus HA is a fifth influenza virus HA, wherein the fifth influenza virus HA is an H1 HA, and wherein the fifth influenza H1 HA is from a different clade than the first influenza H1 HA.

20. The immunogenic composition according to any of claims 1-9, wherein the one or more machine learning influenza virus HAs is a fifth influenza virus HA, wherein the fifth influenza virus HA is an H1 HA, and wherein the fifth influenza H1 HA is antigenically similar to the first influenza H1 HA.

21. The immunogenic composition according to any of claims 1-9, wherein the one or more machine learning influenza virus HAs is a fifth influenza virus HA, wherein the fifth influenza virus HA is an H1 HA, and wherein the fifth influenza H1 HA is from a same clade as the first influenza H1 HA.

22. The immunogenic composition according to any of claims 1-9, wherein the one or more machine learning influenza virus HAs is a fifth influenza virus HA, wherein the fifth influenza virus HA is an H3 HA from the 3C.2A clade.

23. The immunogenic composition according to any of claims 1-9, wherein the one or more machine learning influenza virus HAs is a fifth influenza virus HA, wherein the fifth influenza virus HA is an H3 HA from the 3C.3A clade.

24. The immunogenic composition according to any of claims 1-15, further comprising a sixth influenza virus HA.

25. The immunogenic composition according to claim 24, wherein the sixth influenza virus HA is an H1 HA having a molecular sequence identified or designed from a machine learning model, or a ribonucleic acid molecule encoding the sixth influenza virus HA.

26. The immunogenic composition according to claim 25, wherein the sixth influenza H1 HA is antigenically dissimilar than the first influenza H1 HA, wherein the sixth influenza H1 HA enhances a protective immune response induced by the first influenza H1 HA, wherein the sixth influenza H1 HA broadens a protective immune response induced by the first influenza H1 HA, wherein the sixth influenza H1 HA is from a different clade than the first influenza H1 HA, wherein the sixth influenza H1 HA is from a same clade as the first influenza H1 HA, or wherein the sixth influenza H1 HA is antigenically similar to the first influenza H1 HA.

27. The immunogenic composition according to any one of claims 24-26, further comprising a seventh influenza virus HA from the B/Victoria lineage having a molecular sequence identified or designed from a machine learning model, or a ribonucleic acid molecule encoding the seventh influenza virus HA.

28. The immunogenic composition according to any of claims 24-27, further comprising an eighth influenza virus HA from the B/Yamagata lineage having a molecular sequence identified or designed from a machine learning model, or a ribonucleic acid molecule encoding the eighth influenza virus HA.

29. The immunogenic composition according to any of the preceding claims, wherein the machine learning model is trained to predict a biological response.

30. The immunogenic composition according to claim 29, wherein the biological response is a human, ferret, or mouse biological response.

31. The immunogenic composition according to claim 29 or 30, wherein the biological response comprises a hemagglutinin inhibition assay (HAI), antibody forensics (AF), or neutralization assay.

32. The immunogenic composition according to any one of claims 1-31, wherein each of the first, second, third, and fourth influenza virus HA is a recombinant influenza virus HA.

33. The immunogenic composition according to any one of claims 1-31, wherein each of the first, second, third, and fourth influenza virus HA is present in an inactivated influenza virus.

34. The immunogenic composition according to any one of claims 1-31, comprising the first, second, third, and fourth influenza virus HA as ribonucleic acid molecules.

35. The immunogenic composition according to any one of claims 7-34, wherein each of the recombinant influenza virus HA is produced by a baculovirus expression system in cultured insect cells.

36. The immunogenic composition according to any of the preceding claims, wherein the first influenza virus HA is an H1 HA from an H1N1 influenza virus strain and the second influenza virus HA is an H3 HA from an H3N2 influenza virus strain.

37. The immunogenic composition according to any of the preceding claims, wherein the composition further comprises an adjuvant.

38. The immunogenic composition according to claim 37, wherein the adjuvant comprises a squalene-in-water adjuvant or a liposome-based adjuvant.

39. The immunogenic composition according to claim 38, wherein the squalene-in-water adjuvant comprises AF03.

40. The immunogenic composition according to claim 38, wherein the liposome-based adjuvant comprises SPA14.

41. The immunogenic composition according to any of the preceding claims, wherein each ribonucleic acid molecule comprises one or more modified nucleotides.

42. The immunogenic composition according to any of the preceding claims, wherein the composition is formulated for intramuscular injection.

43. The immunogenic composition according to any of the preceding claims, wherein the ribonucleic acid molecule is encapsulated in an LNP comprising a cationic lipid, a PEGylated lipid, a cholesterol-based lipid, and a helper lipid.

44. A method of immunizing a subject against influenza virus, the method comprising administering to the subject an immunologically effective amount of the immunogenic composition of any one of claims 1-43.

45. The method of claim 44, wherein the method prevents influenza virus infection in the subject.

46. The method of claim 44 or 45, wherein the method raises a protective immune response in the subject.

47. The method of claim 46, wherein the protective immune response comprises an HA antibody response.

48. The method of any one of claims 44-47, wherein the subject is human.

49. The method of any one of claims 44-48, wherein the immunogenic composition is administered intramuscularly, intradermally, subcutaneously, intravenously, intranasally, by inhalation, or intraperitoneally.

50. The method of any one of claims 44-49, wherein the method treats or prevents disease caused by either or both a seasonal and a pandemic influenza strain.

51. The method of any one of claims 44-50, wherein the subject is human and the human is 6 months of age or older, 6 to 35 months of age, at least 2 years of age, at least 3 years of age, less than 18 years of age, at least 18 years of age, at least 60 years of age, at least 65 years of age, at least 6 months of age and less than 18 years of age, at least 3 years of age and less than 18 years of age, or at least 18 years of age and less than 65 years of age.

52. A method of reducing one or more symptoms of influenza virus infection, the method comprising administering to a subject a prophylactically effective amount of the immunogenic composition of any one of claims 1-43.

53. The method of any one of claims 44-52 comprising administering to the subject two doses of the immunogenic composition with an interval of 2-6 weeks, optionally 4 weeks.

54. A vaccine composition comprising the immunogenic composition according to any one of claims 1-43.

55. The method of any one of claims 44-53, wherein the immunogenic composition is a vaccine composition.
